(19) United States
(12) Patent Application Publication
(10) Pub. No.: US 2010/0192250 A1
(43) Pub. Date: Jul. 29, 2010

(54) Use of Fungicides for Making the Phenological Development of Oil Plants More Coherent

(75) Inventor: Herve R. Vantieghem, Stutensee (DE)

Correspondence Address: BRINKS, HOFER, GILSON & LIONE, P.O. BOX 1340, MORRISVILLE, NC, 27560, US

(73) Assignee: BASF SE, Ludwigshafen (DE)

(21) Appl. No.: 12/663,878

(22) PCT Filed: Jun. 12, 2008
(86) PCT No.: PCT/EP2008/057393

§ 371 Date: Dec. 10, 2009

(30) Foreign Application Priority Data
Jun. 12, 2007 (EP) 07110129.9

(51) Int. Cl. A01H 1/06; A01N 43/60; A01N 43/88

(52) U.S. Cl. 800/276; 504/136; 504/130

(57) ABSTRACT

The present invention relates to the use of certain fungicides for obtaining a chronologically more uniform development of oil crops. Furthermore, it relates to a method of increasing the quality and optionally the quantity of oil crop products.

The present invention relates to the use of certain fungicides for obtaining a chronologically more uniform development of oil crops. It also relates to a method of increasing the quality and optionally the quantity of oil crop products.

As a rule, the development within a plant does not proceed in a uniform and homogeneous manner. Thus, the different “storeys” of the plant (i.e. the different, specifically the upper, middle and lower, or the outer and inner, areas of the plant) may flower at different points in time and therefore also form mature fruits/seeds at different points in time. The intervals may amount to several weeks, which makes harvesting considerably more difficult. Since, as a rule, it is neither economically meaningful nor feasible in terms of harvesting technology to harvest repeatedly in a large number of agriculturally important plants, depending on the maturity in individual plant stories, harvesting is generally only done once. Here, however, fruits which are either still immature or already overripe at this point in time may frequently not be utilized, or at least not with a maximum benefit regarding quantity and/or quality. This means that the actual produce yield and/or the quality of the produce yield is markedly lower than what it might be for the plant above.

In oil crops, it happens frequently that oil-comprising fruit/seeds are employed in the further processing, for example oil production, which do not have the ideal degree of maturity, i.e. which are overripe or immature. As a consequence, the quality of the plant products, for example of the oil or its reaction products, may be adversely affected. A high quality of such oil crop products, however, is not only very important in the food and cosmetic sector; high quality standards must also be met when they are used as renewable motor fuels and combustibles.

As a result of the predictable exhaustion of fossil combustibles, the energy sector focuses increasingly on renewable motor fuels and combustibles such as, for example, vegetable oils, biodiesel and bioethanol. Biodiesel refers to the lower-alkyl esters, in particular the methyl esters, of fatty acids. These are obtainable by transesterifying with an alcohol (such as methanol), vegetable oils such as rapeseed oil, but also used fats and used oils, and animal fats which occur naturally in the form of triglycerides. Vegetable oils are, as a rule, obtained by pressing the oil-comprising plant parts of oil crops, for example of oil-comprising fruits or seeds. However, cold-pressing and, in particular, warm-pressing gives an oil which has a relatively high content of phosphorus compounds and mineral compounds, such as alkali metal and in particular alkaline earth metal compounds, mainly calcium compounds and magnesium compounds. These compounds, which are not only present in oil but also in the reaction products thereof, can have an adverse effect on combustion in engines and furnace installations. Moreover, they have a negative effect on the longevity of the material of engines. Negative effects on the exhaust systems can also not be excluded. Thus, the abovementioned compounds result in not inconsiderable ash formation during the combustion operation which puts a strain on, for example, particle filters of diesel vehicles. Nor can the ash be removed by regenerating the particle filter, but it is retained in the filter, which leads to an increased exhaust gas counterpressure. An increased exhaust gas counterpressure leads, in turn, to malfunctions in the diesel engine. In addition, phosphorus compounds act as catalyst poisons and reduce for example the service life of oxidation-type catalytic converters in diesel vehicles and of SCR-type catalytic converters in utility vehicles such as trucks and tractors. Similar problems may also occur in heating installations. To avoid these problems, and also to be able to meet the DIN standard for rapeseed oil as power fuel, which can be expected in the very near future (E DIN 51605), biodiesel or the vegetable oils on which it is based are currently subjected to complicated processing procedures.

Even when using the abovementioned DIN standard for rapeseed oil, it cannot be guaranteed that transport, storage or the combustion of vegetable oils or their reaction products will be problem-free. Thus, certain phosphorus compounds, in particular phospholipids, even if they are present in an amount below the phosphorus limit valve specified by DIN 51605 in the vegetable oil, lead to choking of motor fuel filters in motors, tanks and industrial production plants. It is therefore desirable to reduce the phosphorus content and also the content of other undesirable impurities in the oil even more than specified by DIN 51605.

When using vegetable oils in the food sector and in the cosmetics sector, or when using oil crop products, for example from seeds and presscakes, in the feed sector, too, phosphorus compounds, in particular phosphates, may be a problem for health reasons for example.

Since, in principle, all plant parts such as presscake and seeds may be employed as renewable motor fuels, it is important that these oil crop products have a low phosphorus and mineral content as possible.

Another problem of oil crop products and in particular of vegetable oils and optionally their reaction products is their acid content, which may lead to corrosion in engine and furnace installations, for example in boilers.

It is furthermore desirable to provide vegetable oils and reaction products thereof which have as low an iodine number as possible. The iodine number is then measured for the number of the C—C double bonds in the fatty acid molecules on which the oil or its reaction products is/are based, i.e. for the unsaturated character of the oil. Oils with a higher iodine number are more sensitive to oxidation and therefore become viscous more rapidly than oils with a higher degree of saturation, so that their storage stability is lower. In general, it is desirable to provide vegetable oils or reaction products thereof which have as high an oxidation stability as possible since a sufficient oxidation stability, which is an important aspect of storage stability, is imperative for successful commercialization. The oxidation stability is determined not only by the degree of saturation of the oil, but also by the presence of antioxidants such as vitamin A or vitamin E.

Another problem of vegetable oils, in particular in view of their use in the motor fuel sector, is their viscosity, which is relatively high in comparison with mineral motor fuels. Owing to the poor flow, pumping and atomizing behavior at the fuel injector (droplet spectrum and geometry of the nozzle jet), high viscosity leads to cold-start problems, inter alia. It is therefore desirable to be able to provide vegetable oils with a reduced viscosity, in particular with a reduced kinematic viscosity.

Also desirable are further improvements of the characteristics of oil crop products, in particular of vegetable oils and their reaction products, with regard to their utilization as a source of energy, for example a higher flashpoint, a higher calorific value, a higher cetane number, a lower carbon residue, a reduced sulfur content, a reduced nitrogen content, a reduced chlorine content and a lower content of certain (semi)metal compounds such as zinc, tin, boron and silicon compounds, of oil crop products, especially of vegetable oil or reaction products.

The flashpoint denotes the temperature measured at which vapors emerge in a closed vessel which lead to a vapor/air mixture which is ignitable by an externally supplied ignition force. The flashpoint is used for classifying fluids in hazardous material classes. It is, of course, desirable to provide vegetable oils and reaction products thereof with as high a flashpoint as possible.

The calorific value is a measure for the amount of energy which is liberated upon complete combustion of a substance per volume or per mass. The gross calorific value also contains the energy which is liberated upon condensation of the steam given off upon combustion, while the net calorific value does not include this. Naturally, oil products with as high a net calorific value as possible are desirable.

The cetane number is a measure for the ignition performance of a diesel fuel, and, naturally, motor fuels with good ignition performances are particularly desired.

The carbon residue consists of organic and inorganic material which is generated upon incomplete combustion of motor fuel, and is a measure for the susceptibility of a motor fuel to coking at the fuel injectors and for the formation of residue in the combustion chamber. The coking of fuel injectors leads to a poorer distribution of the injected motor fuel, and thus to reduced engine performance. Coking in motors is currently suppressed especially by addition of specific detergents and dispersants. Naturally, motor fuels with little susceptibility to coking are desirable.

The reduction of the sulfur, nitrogen, chlorine and the abovementioned (semi)metal contents is mainly intended to reduce the discharge of substances which are a health hazard and an environmental hazard, such as sulfuric acid and other sulfur compounds, and nitrose fumes, the reduction of the corrosive effect of oil crop products, mainly vegetable oils and their reaction products, on metal parts which come into contact with them, and the reduction of ash formation, for example as a result of the abovementioned (semi)metal compounds.

The abovementioned quality criteria are influenced, inter alia, by the degree of maturation of the oil crop plant and/or its fruit/seed.

As has already been said above, repeated harvesting in the process of plant maturation in order to ensure that the plant products have as high a quality with regard to the abovementioned criteria as possible, however, not economical, technically difficult to implement as a rule and therefore not common practice; that is to say, as a rule, harvesting is only effected once.

It was therefore an object of the present invention to provide compounds which bring about that the individual development phases within plants, in particular oil crops, proceed more homogeneously in themselves, and therefore within shortened intervals. In particular, the maturation of the fruits/seeds should proceed as homogeneously as possible, i.e. within a shortened interval.

Surprisingly, it has been found that a more homogeneous development of the plant is obtained when the oil crops or their seeds are treated with certain fungicides.

Accordingly, the object is achieved by the use of at least one fungicide selected among aryl- and heterocyclylamides, carbamates, dicarboximides, azoles, strobilurins and morpholines optionally in combination with at least one growth regulator, for achieving a chronologically more uniform development of oil crops.

The chronologically more uniform development of the oil crop refers to a harmonization in comparison with the development of the same oil crop plant (regarding species and variety) under identical growth conditions of the plant, but without treatment of the plant, or its seed, with the specified fungicides.

“Chronologically more uniform development of oil crops” means that individual growth stages of the plant take place in a narrower time window, in particular longitudinal growth, elongation and, especially, flowering and/or maturation of the fruit/seed.

The use according to the invention of the specified fungicides preferably bring about a longitudinal growth and/or elongation and/or flowering within the plant and/or maturation of the fruit/seed of the plant within a more uniform interval, i.e. a narrower interval, in comparison with plants which have not been treated in accordance with the invention.

Especially preferably, flowering within the plant and/or maturation of the fruit/seed of the plant takes place within a more uniform interval, i.e. a narrower interval, in comparison with plants which have not been treated in accordance with the invention. In particular maturation of the fruit/seed of the plant takes place within a more uniform time frame, i.e. a narrower interval, in comparison with plants which have not been treated in accordance with the invention.

“Within the plant” means that the development of one and the same plant takes place in a more concentrated fashion.

Oil crops are plants whose plant parts, in particular whose fruits and/or seeds, yield oil. They can be divided into two main groups:
fruit pulp oil crops, where the oil is obtained from the fatty fruit pulp. These include, for example, olive trees and oil palms.
Seed oil crops, where the oil is obtained from the seeds. These include, for example, oilseed rape, turnip rape, mustard, oil radish, false flax, garden rocket, crambe, sunflower, safflower, thistle, calendula, soybean, lupine, flax, hemp, oil pumpkin, poppy, maize and nuts, in particular Arachids (peanuts).

The two species mentioned above for the fruit pulp oil crops (olive tree and oil palm) can, however, also be included in the seed oil crops, since the seed (stone) of both is likewise used for obtaining oil.

Preferred oil crops are seed oil crops in the stricter sense, i.e. oil crops which have no additional, oil-comprising fruit pulp.

For the purposes of the present invention, the terms “fruit” and “seed”, on which the definition of the terms “fruit pulp oil crops” and “seed oil crops” is based, are not used in the strict morphological sense, i.e. no differentiation is made on the basis of the flower parts from which the seed or the fruit develops. Rather, the term “seed” is understood as meaning, for the purposes of the present invention, the part of the plant which can be used as such, i.e. without further processing, as seed. The fruit, in contrast, is the totality of the organs which develop from a flower and which enclose the seeds until they are mature. A fruit comprises one or more seeds which are surrounded by the pericarp. For the purposes of the present invention, a fruit additionally comprises fruit pulp, which can readily be separated from the seed in the morphological sense. Moreover, in the case of a fruit for the purposes of the invention, the pericarp is not inseparably fused with the seed or the seed coat. Seed oil crops for the purposes of the invention thus comprise not only oil crops where the oil is obtained from seeds in the morphological sense, but also oil crops in which the oil is obtained from the kind of fruit where the pericarp is inseparably fused with the seed, as is the case for example in sunflowers, nuts or maize. Accordingly, for the purposes of the present invention, the term “seed coat” is not limited to the coat of seeds in the morphological sense, but also comprises the pericarps of fruits where the pericarp is inseparably fused with the seed and which thus come under the term “seeds” as used in accordance with the invention.

Preferably, however, the term “fruit/seed” is understood to mean the seed without detachable fruit pulp.

Furthermore, the invention relates to a method of increasing the quality and optionally the quantity of oil crop products, in which a (live) oil crop plant or (live) plant part thereof or their seed (i.e. the seed from which the plant grows) is treated with at least one fungicide, optionally in combination with at least one growth regulator, as defined hereinabove, the fruit/seed of the oil crop plant are harvested when their water content amounts to no more than 15% by weight based on the total weight of the fruit/seed, as the oil crop product is obtained, the increase in quality being selected among the following criteria:
(i) reducing the phosphorus content of at least one oil crop product;
(ii) reducing the alkali and/or alkaline earth metal content of at least one oil crop product;
(iii) increasing the oxidation stability of at least one oil crop product;
(iv) reducing the overall contamination of at least one oil crop product;
(v) lowering the iodine number of at least one oil crop product;
(vi) lowering the acid number of at least one oil crop product;
(vii) reducing the kinematic viscosity of at least one oil crop product;
(viii) reducing the sulfuric content of at least one oil crop product;
(ix) increasing the flashpoint of at least one oil crop product;
(x) increasing the net calorific value of at least one oil crop product;
(xi) reducing the carbon residue of at least one oil crop product;
(xii) increasing the cetane number of at least one oil crop product;
(xiii) reducing the nitrogen content of at least one oil crop product;
(xiv) reducing the chlorine content of at least one oil crop product; and
(xv) reducing the tin, zinc, silicon and/or boron content of at least one oil crop product.

Those criteria which are not improved by individual treatments according to the invention are, however, preferably also not made worse.

An increase in quality and optionally an increase in quantity of the at least one oil crop product relates to an improvement in comparison with the quality and optionally quantity of the same oil crop product which has been obtained, in the same manner (regarding harvesting, processing and the like), from the same oil crop plant (regarding species and variety) under identical growth conditions of the plant, but without the treatment of the plant or its seed with the specified fungicides and/or without harvest at the described point in time.

For the purposes of the present invention, oil crop products are understood as meaning all oil-comprising plant parts of oil crops, their processed products and reaction products, and the reaction products of the processed products. They are suitable as a source of energy, for example in the form of combustibles and motor fuels, as lubricants, but also for use in the food and feed sector, or else in the cosmetics sector. The oil crop products include mainly the oil-comprising fruits and seeds of oil crops, the oil obtained therefrom (which can be employed in the food sector, for example as edible oil or for the production of margarine, in the cosmetics sector, for example as carrier, as lubricant or as combustible and motor fuel), the presscake obtained during the pressing process upon oil extraction (which can be employed in the feed sector as animal feed, or as combustible) and the reaction products of the oil, for example its transesterification products with C₁-C₄-alcohols, preferably with methanol (which can be employed as biodiesel). Transesterification products of the oil with C₁-C₄ alcohols are understood as meaning the C₁-C₄ alkyl esters of the fatty acids present in the oil, principally as glycerides (especially as triglycerides).

The oil crop products are preferably selected among vegetable oils and their reaction products, for example the transesterification products with C₁-C₄-alcohols, preferably with methanol.

For the purpose of the present invention, oils are understood as meaning vegetable oils, unless otherwise specified.

For the purposes of the present invention, the generic terms used have the following meanings:

Halogen is fluorine, chlorine, bromine or iodine, in particular fluorine, chlorine or bromine.

The term “partially or fully halogenated” means that one or more, for example 1, 2, 3 or 4 or all hydrogen atoms of a particular radical are replaced by halogen atoms, in particular by fluorine or chlorine.

The term “Cₘ—Cₙ-alkyl” (also in Cₘ-Cₙ-haloalkyl, Cₘ-Cₙ-alkylthio, Cₘ-Cₙ-haloalkylthio, Cₘ-Cₙ-alkylsulfinyl and Cₘ-Cₙ-alkylsulfonyl) is a linear or branched saturated hydrocarbon radical having m to n, for example 1 to 8, carbon atoms. Thus, C₁-C₄-alkyl is, for example, methyl, ethyl, propyl, isopropyl, n-butyl, sec-butyl, isobutyl or tert-butyl. C₁-C₈-Alkyl is, additionally, for example pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, heptyl, octyl, 2-ethylhexyl, and their constitutional isomers.

Cₘ-Cₙ-Haloalkyl is a linear or branched alkyl radical having m to n carbon atoms in which one or more hydrogen atoms are replaced by halogen atoms, in particular fluorine or chlorine. Thus, C₁-C₈-haloalkyl is a linear or branched C₁-C₈-alkyl radical in which one or more hydrogen atoms are replaced by halogen atoms, in particular fluorine or chlorine. C₁-C₈-Haloalkyl is, in particular, C₁-C₂-haloalkyl. C₁-C₂-Haloalkyl is, for example, chloromethyl, dichloromethyl, trichloromethyl, bromomethyl, fluoromethyl, difluoromethyl, trifluoromethyl, chlorofluoromethyl, dichlorofluoromethyl, chlorodifluoromethyl, 1-chloroethyl, 2-chloroethyl, 1-fluoroethyl, 2-fluoroethyl, 2,2-difluoroethyl, 2,2,2-trifluoroethyl, pentafluoroethyl and the like.

Cₘ-Cₙ-Alkoxy is a linear or branched alkyl radical having m to n carbon atoms which is bonded via an oxygen atom. Accordingly, C₁-C₄-alkoxy is a C₁-C₄-alkyl radical which is bonded via an oxygen atom. Examples are methoxy, ethoxy, propoxy, isopropoxy, butoxy, sec-butoxy, isobutoxy and tert-butoxy. Examples of C₁-C₈-alkoxy are, additionally, pentyloxy, hexyloxy, octyloxy and their constitutional isomers. C₁-C₈-Haloalkoxy is a linear or branched C₁-C₈-alkyl radical which is bonded via an oxygen atom and in which one or more hydrogen atoms are replaced by a halogen atom, in particular by fluorine or chlorine. Examples are chloromethoxy, dichloromethoxy, trichloromethoxy, fluoromethoxy, difluoromethoxy, trifluoromethoxy, bromomethoxy, chlorofluoromethoxy, dichlorofluoromethoxy, chlorodifluoromethoxy, 1-chloroethoxy, 1-bromoethoxy, 1-fluoroethoxy, 2-chloroethoxy, 2-bromoethoxy, 2-fluoroethoxy, 2,2-difluoroethoxy, 2-chloro-2-fluoroethoxy, 2,2-dichloroethoxy, 2,2,2-trichloroethoxy, 2,2,2-trifluoroethoxy, pentafluoroethoxy, pentachloroethoxy and the like.

C₁-C₈-Alkylthio, C₁-C₈-alkylsulfinyl and C₁-C₈-alkylsulfonyl are a linear or branched C₁-C₈-alkyl radical which is bonded via a sulfur atom (alkylthio), an S(O) group (alkylsulfinyl) or an S(O)₂ group (alkylsulfonyl). Examples of C₁-C₈-alkylthio comprise methylthio, ethylthio, propylthio, isopropylthio, n-butylthio and the like. Examples of C₁-C₈-alkylsulfinyl comprise methylsulfinyl, ethylsulfinyl, propylsulfinyl, isopropylsulfinyl, n-butylsulfinyl and the like. Examples of C₁-C₈-alkylsulfonyl comprise methylsulfonyl, ethylsulfonyl, propylsulfonyl, isopropylsulfonyl, n-butylsulfonyl and the like.

C₁-C₄-Alkylthio is a linear or branched C₁-C₈-alkyl radical which is bonded via a sulfur atom. Examples comprise methylthio, ethylthio, propylthio, isopropylthio, n-butylthio and their constitutional isomers.

C₁-C₈-Haloalkylthio is a linear or branched C₁-C₈-alkyl radical which is bonded via a sulfur atom and in which one or more hydrogen atoms are replaced by a halogen atom, in particular by fluorine or chlorine. Examples are chloromethylthio, dichloromethylthio, trichloromethylthio, fluoromethylthio, difluoromethylthio, trifluoromethylthio, bromomethylthio, chlorofluoromethylthio, dichlorofluoromethylthio, chlorodifluoromethylthio, 1-chloroethylthio, 1-bromoethylthio, 1-fluoroethylthio, 2-chloroethylthio, 2-bromoethylthio, 2-fluoroethylthio, 2,2-difluoroethylthio, 2-chloro-2-fluoroethylthio, 2,2-dichloroethylthio, 2,2,2-trichloroethylthio, 2,2,2-trifluoroethylthio, pentafluoroethylthio, pentachloroethylthio and the like.

Cₘ-Cₙ-Alkoxy-Cₘ-Cₙ-alkyl is a Cₘ-Cₙ-alkyl group in which one hydrogen atom is replaced by a Cₘ-Cₙ-alkoxy group. Accordingly, C₁-C₈-alkoxy-C₁-C₈-alkyl is a C₁-C₈-alkyl group in which one hydrogen atom is replaced by a C₁-C₈-alkoxy group. Examples are methoxymethyl, ethoxymethyl, propoxymethyl, methoxyethyl, ethoxyethyl, propoxyethyl, methoxypropyl, ethoxypropyl, propoxypropyl and the like.

Cₘ-Cₙ-Alkylthio-Cₘ-Cₙ-alkyl is a Cₘ-Cₙ-alkyl group in which one hydrogen atom is replaced by a Cₘ-Cₙ-alkylthio group. Accordingly, C₁-C₈-alkylthio-C₁-C₈-alkyl is a C₁-C₈-alkyl group in which one hydrogen atom is replaced by a C₁-C₈-alkylthio group. Examples are methylthiomethyl, ethylthiomethyl, propylthiomethyl, methylthioethyl, ethylthioethyl, propylthiomethyl, methylthiopropyl, ethylthiopropyl, propylthiopropyl and the like.

Cₘ-Cₙ-Haloalkylthio-Cₘ-Cₙ-alkyl is a Cₘ-Cₙ-alkyl group in which one hydrogen atom is replaced by a Cₘ-Cₙ-haloalkylthio group. Accordingly, C₁-C₈-haloalkylthio-C₁-C₈-alkyl is a C₁-C₈-alkyl group in which one hydrogen atom is replaced by a C₁-C₈-haloalkylthio group. Examples are chloromethylthiomethyl, dichloromethylthiomethyl, trichloromethylthiomethyl, chloroethylthiomethyl, dichloroethylthiomethyl, trichloroethylthiomethyl, tetrachloroethylthiomethyl, pentachloroethylthiomethyl and the like.

Carboxyl is a group —COOH.

C₁-C₈-Alkylcarbonyl is a group —CO—R in which R is C₁-C₈-alkyl.

C₁-C₈-Alkyloxycarbonyl (also referred to as C₁-C₈-alkoxycarbonyl) is a group —C(O)O—R in which R is C₁-C₈-alkyl.

C₁-C₈-Alkylcarbonyloxy is a group —OC(O)—R in which R is C₁-C₈-alkyl.

C₁-C₈-Alkylaminocarbonyl is a group —CO—NH—R in which R is C₁-C₈-alkyl.

Di(C₁-C₈-alkyl)aminocarbonyl is a group —CO—N(RR′) in which R and R′, independently of one another, are C₁-C₈-alkyl.

C₂-C₈-Alkenyl is a linear or branched hydrocarbon having 2 to 8 carbon atoms and one double bond in any position. Examples are ethenyl, 1-propenyl, 2-propenyl (allyl), 1-methylethenyl, 1-, 2- and 3-butenyl, 1-methyl-1-propenyl, 2-methyl-1-propenyl, 1-, 2-, 3- and 4-pentenyl, 1-, 2-, 3-, 4- and 5-hexenyl, 1-, 2-, 3-, 4-, 5- and 6-heptenyl, 1-, 2-, 3-, 4-, 5-, 6- and 7-octenyl and their constitutional isomers.

C₂-C₈-Alkenyloxy is a C₂-C₈-alkenyl radical which is bonded via an oxygen atom. Examples are ethenyloxy, propenyloxy and the like.

C₂-C₈-Alkenylthio is a C₂-C₈-alkenyl radical which is bonded via a sulfur atom. Examples are ethenylthio, propenylthio and the like.

C₂-C₈-Alkenylamino is a group —NH—R in which R is C₂-C₈-alkenyl.

N—C₂-C₈-Alkenyl-N—C₁-C₈-alkylamino is a group —N(RR′) in which R is C₂-C₈-alkenyl and R′ is C₁-C₈-alkyl.

C₂-C₈-Alkynyl is a linear or branched hydrocarbon having 2 to 8 carbon atoms and at least one triple bond. Examples are ethynyl, propynyl, 1- and 2-butynyl and the like.

C₂-C₈-Alkynyloxy is a C₂-C₈-alkynyl radical which is bonded via an oxygen atom. Examples are propynyloxy, butynyloxy and the like.

C₂-C₈-Alkynylthio is a C₂-C₈-alkynyl radical which is bonded via a sulfur atom. Examples are ethenylthio, propynylthio and the like.

C₂-C₈-Alkynylamino is a group —NH—R in which R is C₂-C₈-alkynyl.

N—C₂-C₈-Alkynyl-N—C₁-C₈-alkylamino is a group —N(RR′) in which R is C₂-C₈-alkynyl and R′ is C₁-C₈-alkyl.

C₃-C₈-Cycloalkyl is a monocyclic 3- to 8-membered saturated cycloaliphatic radical. Examples are cyclopropyl, cyclobutyl, cyclopentyl, cyclohexyl, cycloheptyl and cyclooctyl. C₃-C₁₀-Cycloalkyl is a monocyclic 3- to 10-membered saturated cycloaliphatic radical. Examples are cyclononyl and cyclodecyl, in addition to the radicals mentioned for C₃-C₈-cycloalkyl.

C₃-C₈-Cycloalkyloxy (or C₃-C₈-cycloalkoxy) is a C₃-C₈-cycloalkyl radical which is bonded via oxygen. Examples are cyclopropyloxy, cyclobutyloxy, cyclopentyloxy, cyclohexyloxy, cycloheptyloxy and cyclooctyloxy.

C₃-C₈-Cycloalkylthio is a C₃-C₈-cycloalkyl radical which is bonded via a sulfur atom. Examples are cyclopropylthio, cyclobutylthio, cyclopentylthio, cyclohexylthio, cycloheptylthio and cyclooctylthio.

C₃-C₈-Cycloalkylamino is a group —NH—R in which R is C₃-C₈-cycloalkyl.

N—C₃-C₈-Cycloalkyl-N—C₁-C₈-alkylamino is a group N(RR′) in which R is C₃-C₈-cycloalkyl and R′ is C₁-C₈-alkyl.

C₃-C₈-Cycloalkenyl is a monocyclic 3- to 8-membered unsaturated cycloaliphatic radical having at least one double bond. Examples are cyclopropenyl, cyclobutenyl, cyclopentenyl, cyclopentadienyl, cyclohexyl, cyclohexadienyl, cycloheptenyl, cycloheptadienyl, cyclooctyl, cyclooctadienyl, cyclooctatrienyl and cyclooctatetraenyl.

C₃-C₈-Cycloalkenyloxy is a C₃-C₈-cycloalkenyl radical which is bonded via oxygen. Examples are cyclopropenyloxy, cyclobutenyloxy, cyclopentenyloxy, cyclopentadienyloxy, cyclohexenyloxy, cyclohexadienyloxy, cycloheptenyloxy, cycloheptadienyloxy, cyclooctenyloxy, cyclooctadienyloxy, cyclooctatrienyloxy and cyclooctatetraenyloxy.

Cₘ-Cₙ-Alkylene is a linear or branched alkylene group having m to n, for example 1 to 8, carbon atoms. Thus, C₁-C₃-alkylene is, for example, methylene, 1,1- or 1,2-ethylene, 1,1-, 1,2-, 2,2- or 1,3-propylene. C₂-C₄-Alkylene is, for example, 1,1- or 1,2-ethylene, 1,1-, 1,2-, 2,2- or 1,3-propylene, 1,1-, 1,2-, 1,3- or 1,4-butylene. C₃-C₅-Alkylene is, for example, 1,1-, 1,2-, 2,2- or 1,3-propylene, 1,1-, 1,2-, 1,3- or 1,4-butylene, 1,1-dimethyl-1,2-ethylene, 2,2-dimethyl-1,2-ethylene, 1,1-, 1,2-, 1,3-, 1,4- or 1,5-pentylene and the like.

Oxy-Cₘ-Cₙ-alkylene is a group —O—R— in which R is Cₘ-Cₙ-alkylene. Thus, oxy-C₂-C₄-alkylene is a group —O—R— in which R is C₂-C₄-alkylene. Examples are oxyethylene, oxypropylene and the like.

Oxy-Cₘ-Cₙ-alkylenoxy is a group —O—R—O— in which R is Cₘ-Cₙ-alkylene. Thus, oxy-C₂-C₄-alkylenoxy is a group —O—R—O— in which R is C₁-C₃-alkylene. Examples are oxymethylenoxy, oxy-1,2-ethylenoxy, oxy-1,3-propylenoxy and the like.

Cₘ-Cₙ-Alkenylene is a linear or branched alkenylene group having m to n, for example 2 to 8, carbon atoms and a C—C double bond at any position. Thus, C₂-C₄-alkenylene is, for example, 1,1- or 1,2-ethenylene, 1,1-, 1,2- or 1,3-propenylene, 1,1-, 1,2-, 1,3- or 1,4-butylene. C₃-C₅-Alkenylene is, for example, 1,1-, 1,2- or 1,3-propenylene, 1,1-, 1,2-, 1,3- or 1,4-butenylene, 1,1-, 1,2-, 1,3-, 1,4- or 1,5-pentenylene and the like.

Oxy-Cₘ-Cₙ-alkenylene is a group —O—R— in which R is Cₘ-Cₙ-alkenylene. Thus, oxy-C₂-C₄-alkenylene is a group —O—R— in which R is C₂-C₄-alkenylene. Examples are oxyethenylene, oxypropenylene and the like.

Oxy-Cₘ-Cₙ-alkenylenoxy is a group —O—R—O— in which R is Cₘ-Cₙ-alkenylene. Thus, oxy-C₂-C₄-alkenylenoxy is a group —O—R—O— in which R is C₂-C₄-alkenylene. Examples are oxyethenylenoxy, oxypropenylenoxy and the like.

Cₘ-Cₙ-Alkynylene is a linear or branched alkynylene group having m to n, for example 2 to 8, carbon atoms and a C—C triple bond at any position. Thus, C₂-C₄-alkynylene is, for example, 1,1- or 1,2-ethynylene, 1,1-, 1,2- or 1,3-propynylene, 1,1-, 1,2-, 1,3- or 1,4-butynylene. C₃-C₅-Alkynylene is, for example, 1,1-, 1,2- or 1,3-propynylene, 1,1-, 1,2-, 1,3- or 1,4-butynylene, 1,1-, 1,2-, 1,3-, 1,4- or 1,5-pentynylene and the like.

C₁-C₄-Alkanols (═C₁-C₄-alcohols) are, for the purposes of the present invention, aliphatic C₁-C₄-hydrocarbons in which one hydrogen atom is replaced by a hydroxyl group. Examples are methanol, ethanol, propanol, isopropanol, n-butanol, sec-butanol, isobutanol and tert-butanol.

Aryl is an optionally substituted aromatic hydrocarbon radical having 6 to 14 carbon atoms, such as phenyl, naphthyl, anthracenyl or phenanthrenyl and in particular phenyl. Examples of suitable substituents are halogen, C₁-C₈-alkyl, C₁-C₈-alkoxy, OH, NO₂, CN, COOH, C₁-C₈-alkylcarbonyl, C₁-C₈-alkylcarbonyloxy, C₁-C₈-alkyloxycarbonyl, NH₂, C₁-C₈-alkylamino, di(C₁-C₈-alkyl)amino and other substituents which are mentioned hereinbelow.

Aryloxy is an aryl radical which is bonded via an oxygen atom. An example is optionally substituted phenoxy.

Arylthio is an aryl radical which is bonded via a sulfur atom. An example is optionally substituted phenylthio.

Aryl-C₁-C₈-alkyl is a C₁-C₈-alkyl radical in which one hydrogen atom is substituted by an aryl group. Examples are benzyl and 2-phenylethyl.

Aryl-C₂-C₈-alkenyl is a C₂-C₈-alkenyl radical in which one hydrogen atom is substituted by an aryl group. An example is 2-phenylethenyl (styryl).

Aryl-C₂-C₈-alkynyl is a C₂-C₈-alkynyl radical in which one hydrogen atom is substituted by an aryl group. An example is 2-phenylethynyl.

Aryl-C₁-C₈-alkoxy is a C₁-C₈-alkoxy radical in which one hydrogen atom is replaced by an aryl group.

Arylthio-C₁-C₄-alkyl is a C₁-C₄-alkyl radical in which one hydrogen atom is substituted by an aryl group, for example optionally substituted phenylthio-C₁-C₄-alkyl. Examples of optionally substituted phenylthio-C₁-C₄-alkyl are phenylthiomethyl (C₆H₅—S—CH₂) and phenylthioethyl (C₆H₅—S—CH₂CH₂), it being possible for the phenyl radical to be substituted, for example by one or more chlorine atoms.

Heterocyclyl is a nonaromatic saturated or unsaturated or aromatic (“hetaryl”) heterocyclyl radical having preferably 3 to 7 ring members and 1, 2, 3 or 4 hetero atoms selected from among O, N and S and/or hetero atom groups selected from among SO, SO₂ and NR, where R is H or C₁-C₈-alkyl as ring members and furthermore optionally 1, 2 or 3 carbonyl groups as ring members. Examples of nonaromatic heterocyclyl groups comprise aziridinyl, azetidinyl, pyrrolidinyl, pyrrolidinonyl, pyrrolidinedionyl, pyrazolinyl, pyrazolinonyl, imidazolinyl, imidazolinonyl, imidazolinedionyl, pyrrolinyl, pyrrolinonyl, pyrrolinedionyl, pyrazolinyl, imidazolinyl, imidazolinonyl, tetrahydrofuranyl, dihydrofuranyl, 1,3-dioxolanyl, dioxolenyl, thiolanyl, dihydrothienyl, oxazolidinyl, isoxazolidinyl, oxazolinyl, isoxazolinyl, thiazolinyl, isothiazolinyl, thiazolidinyl, isothiazolidinyl, oxathiolanyl, piperidinyl, piperidinonyl, piperidinedionyl, piperazinyl, pyridinonyl, pyridinedionyl, pyridazinonyl, pyridazinedionyl, pyrimidinonyl, pyridazinedionyl, pyranyl, dihydropyranyl, tetrahydropyranyl, dioxanyl, thiopyranyl, dihydrothiopyranyl, tetrahydrothiopyranyl, morpholinyl, thiazinyl and the like. Examples of aromatic heterocyclyl groups (hetaryl) comprise pyrrolyl, furyl, thienyl, pyrazolyl, imidazolyl, triazolyl, tetrazolyl, oxazolyl, isoxazolyl, thiazolyl, isothiazolyl, thiadiazolyl, oxadiazolyl, pyridyl, pyridazinyl, pyrimidinyl, pyrazinyl and triazinyl.

Heterocyclyloxy or hetaryloxy is a heterocyclyl, or hetaryl, radical which is bonded via an oxygen atom.

Hetaryl-C₁-C₈-alkyl is a C₁-C₈-alkyl radical in which one hydrogen atom is substituted by a hetaryl group. Examples are pyrrolylmethyl, pyridinylmethyl and the like.

Hetaryl-C₂-C₈-alkenyl is a C₂-C₈-alkenyl radical in which one hydrogen atom is substituted by a hetaryl group.

Hetaryl-C₂-C₈-alkynyl is a C₂-C₈-alkynyl radical in which one hydrogen atom is substituted by a hetaryl group.

Hetaryl-C₁-C₈-alkoxy is a C₁-C₈-alkoxy radical in which one hydrogen atom is substituted by a hetaryl group.

The above and the following observations made with regard to preferred features of the invention apply by themselves, but also in combination with other preferred features.

“Increase in quality” means preferably that at least one oil crop product must meet at least one of the criteria (i) to (xi), more preferably (i) to (viii), even more preferably (i) to (vii), in particular (i) to (iii) and (vi), specifically (i), (ii) or (vi), and more specifically (i) or (vi).

Examples of suitable oil crops are oilseed rape, turnip rape, mustard, oil radish, false flax, garden rocket, crambe, sunflower, safflower, thistle, calendula, soybean, lupine, flax, hemp, oil pumpkin, poppy, maize, oil palm and peanut.

The oil crops are preferably selected among seed oil crops in the stricter sense.

Seed oil crops are preferably selected among oilseed rape, turnip rape, mustard, oil radish, false flax, garden rocket, crambe, sunflower, safflower, thistle, calendula, soybean, lupine, flax, hemp, oil pumpkin, poppy and maize.

The oil crops/seed oil crops are especially preferably selected among oilseed rape, turnip rape, sunflower, soybean, flax and maize, more preferably among oilseed rape, turnip rape and sunflower, even more preferably among oilseed rape and turnip rape, and in particular oilseed rape.

Preferred in particular for an application in the food and feed sector is 0 oilseed rape and, in particular, 00 oilseed rape. Other types of oilseed rape, for example varieties comprising erucic acid and glucosinolate, are also suitable for other applications.

The fungicides employed in accordance with the invention are selected among aryl- and heterocyclylamides (hereinbelow also referred to as amide fungicides), carbamates, dicarboximides, azoles, strobilurin and morpholine. In one embodiment of the invention, the fungicides employed are selected among aryl- and heterocyclylamides, carbamates, dicarboximides, azoles and strobilurin. Preferably, the fungicides employed in accordance with the invention are selected among aryl- and heterocyclylamides, strobilurins and azoles. Especially preferably, the fungicides employed in accordance with the invention are selected among aryl- and hetero-cyclylamides and azoles. Specifically, at least one aryl- or heterocyclylamide is used in combination with at least one azole.

Aryl- and heterocyclylamides (amide fungicides) are understood as meaning fungicides which comprise a carboxamide group in which the amine moiety is derived from optionally substituted aniline or from an optionally substituted hetarylamine and the carbonyl group has attached to it an optionally substituted aryl- or heterocyclyl radical.

Amide fungicides, which are also referred to as carboxamide fungicides or, specifically for the case where the amine moiety is derived from aniline, as anilide fungicide, and processes for their preparation are known to the skilled worker in principle and are described for example in Farm Chemicals Handbook, Meister Publishing Company or in the Compendium of Pesticide Common Names, http://www.hclrss.demon.co.uk/, hereby fully incorporated herein by reference.

Preferred amide fungicides are those of the formula I

A-CO—NH-M-Q-R¹

in which
A is an aryl group or an aromatic or nonaromatic 5- or 6-membered heterocycle which comprises, as ring members, 1 to 3 heteroatoms or heteroatom-comprising groups selected among O, S, N and NR², R² being hydrogen or C₁-C₈-alkyl, the aryl group or the heterocycle optionally having 1, 2 or 3 substituents which are selected independently of one another among halogen, C₁-C₈-alkyl, C₁-C₈-haloalkyl, C₁-C₈-alkoxy, C₁-C₈-haloalkoxy, C₁-C₈-alkylthio, C₁-C₈-alkylsulfinyl and C₁-C₈-alkylsulfonyl;
M is a thienyl ring or a phenyl ring, where the thienyl and the phenyl ring may have attached to them 1, 2 or 3 halogen atoms and where the phenyl ring is optionally fused to a saturated 5-membered ring which is optionally substituted by 1, 2 or 3 C₁-C₈-alkyl groups and/or optionally contains, as ring member, a hetero atom selected among O and S;
Q is a bond, C₁-C₆-alkylene, C₂-C₆-alkenylene, C₂-C₆-alkynylene, C₃-C₆-cycloalkylene, C₃-C₆-cycloalkenylene, —O—C₁-C₆-alkylene, —O—C₂-C₆-alkenylene, —O—C₂-C₆-alkynylene, —O—C₃-C₆-cycloalkylene, —O—C₃-C₆-cycloalkenylene, —S—C₁-C₆-alkylene, —S—C₂-C₆-alkenylene, —S—C₂-C₆-alkynylene, —S—C₃-C₆-cycloalkylene, —S—C₃-C₆-cycloalkenylene, —SO—C₁-C₆-alkylene, —SO—C₂-C₆-alkenylene, —SO—C₂-C₆-alkynylene, —SO—C₃-C₆-cycloalkylene, —SO—C₃-C₆-cycloalkenylene, —SO₂—C₁-C₆-alkylene, —SO₂—C₂-C₆-alkenylene, —SO₂—C₂-C₆-alkynylene, —SO₂—C₃-C₆-cycloalkylene, —SO₂—C₃-C₆-cycloalkenylene, O, S, SO or SO₂;
   where the aliphatic and cycloaliphatic radical in Q may be partially or fully halogenated and/or the cycloaliphatic radical may be substituted by 1, 2 or 3 C₁-C₈-alkyl radicals;
R¹ is hydrogen, halogen, C₃-C₆-cycloalkyl or phenyl, where the cycloalkyl radical may have attached to it a methyl group and where phenyl may be substituted by 1 to 5 halogen atoms and/or by 1, 2 or 3 substituents which are selected independently of one another among C₁-C₈-alkyl, C₁-C₈-alkoxy, C₁-C₈-haloalkoxy, C₁-C₈-alkylthio and C₁-C₈-haloalkylthio.

Amides of the formula I and processes for their preparation are known per se and described, for example, in EP-A-545099, EP-A-589301, EP-A 737682, EP-A 824099, WO 97/08952, WO 99/09013, WO 03/010149, WO 03/070705, WO 03/074491, WO 2004/005242 and WO 2004/067515 and in the literature cited therein, hereby fully incorporated herein by reference.

The carboxamide group and the radical Q are preferably bonded to adjacent carbon atoms of the radical M.

In a preferred embodiment, Q is a single bond and R¹ is hydrogen.

In an alternatively preferred embodiment, Q is a single bond and R¹ is phenyl which is substituted by 1, 2 or 3 hydrogen atoms.

In an alternatively preferred embodiment, Q is C₁-C₆-alkylene and R¹ is hydrogen.

In an alternatively preferred embodiment, Q and R¹ together form —O—C₁-C₄-haloalkyl or —S—C₁-C₄-haloalkyl.

In an alternatively preferred embodiment, Q is cyclopropylene and R¹ is cyclopropyl which optionally has a methyl group attached to it. Preferably, the two rings are substituted in the trans position.

A is preferably selected among radicals of the formulae (A1) to (A8) referred to hereinbelow and especially preferably among radicals of the formulae (A1), (A2), (A5) and (A7) described hereinbelow.

In a preferred embodiment, M is thienyl.

In an alternatively preferred embodiment, M is phenyl. In this case, M preferably has attached to it the radical Q-R¹ as the only substituent. Alternatively preferably, M has attached to it in addition to the radical Q-R¹, a halogen atom, where fluorine is preferred. Preferably, the halogen atom is bonded in the para position relative to the carboxamide group.

The amide of the formula I is especially preferably selected among anilides of the formula I.1

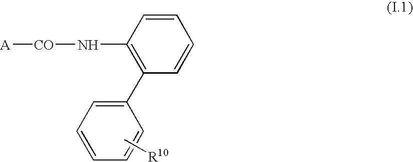

in which A is a group of the formula A1 to A8

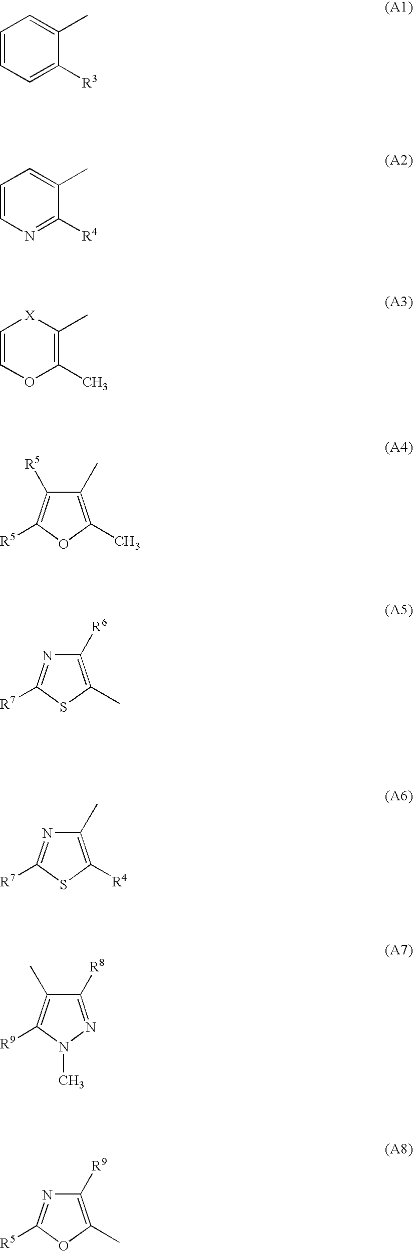

in which
X is CH₂, S, SO or SO₂;
R³ is CH₃, CHF₂, CF₃, Cl, Br or I;
R⁴ is CF₃ or Cl;
R⁵ is hydrogen or CH₃;
R⁶ is CH₃, CHF₂, CF₃ or Cl;
R⁷ is hydrogen, CH₃ or Cl;
R⁸ is CH₃, CHF₂ or CF₃;
R⁹ is hydrogen, CH₃, CHF₂, CF₃ or Cl; and
R¹⁰ is C₁-C₄-alkyl, C₁-C₄-alkoxy, C₁-C₄-alkylthio or halogen.

Group A is preferably the group A2 in which R⁴ is halogen. Preferably, R¹⁰ is simultaneously halogen.

In particular, the amide fungicide of the formula I is selected among anilides of the formula I.1.1 and I.1.2

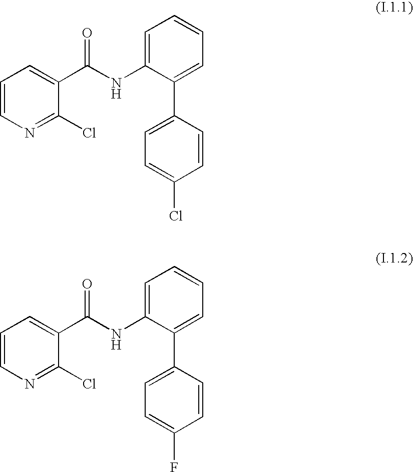

Among these, the anilide I.1.1 is especially preferred. This compound is also known under its common name boscalid and commercially available.

Alternatively preferred are amides I in which A is a radical of the formula (A1) to (A8), M is phenyl or thienyl, Q is C₁-C₆-alkylene and R¹ is hydrogen.

Alternatively preferred are amides I in which A is a radical of the formula (A1) to (A8), M is phenyl, Q is cyclopropylene and R¹ is cyclopropyl which optionally has a methyl group attached to it. Preferably, both rings are substituted in the trans position.

With regards the anilide (I.1), in particular (I.1.1) and (I.1.2), especially preferred compounds are selected among:
2-iodo-N-phenylbenzamide, 2-chloro-N-(4′-chlorobiphenyl-2-yl)nicotinamide,
N-[2-(1,3-dimethylbutyl)thiophen-3-yl]-3-trifluoromethyl-1-methylpyrazol-4-ylcarboxamide,
N-(2-bicyclopropyl-2-ylphenyl)-3-difluoromethyl-1-methylpyrazol-4-ylcarboxamide,
N-(3′,4′,5′-trifluorobiphenyl-2-yl)-1,3-dimethylpyrazol-4-ylcarboxamide,
N-(3′,4′,5′-trifluorobiphenyl-2-yl)-1,3-dimethyl-5-fluoropyrazol-4-ylcarboxamide,
N-(3′,4′,5′-trifluorobiphenyl-2-yl)-5-chloro-1,3-dimethylpyrazol-4-ylcarboxamide,
N-(3′,4′,5′-trifluorobiphenyl-2-yl)-3-fluoromethyl-1-methylpyrazol-4-ylcarboxamide,
N-(3′,4′,5′-trifluorobiphenyl-2-yl)-3-(chlorofluoromethyl)-1-methylpyrazol-4-ylcarboxamide,
N-(3′,4′,5′-trifluorobiphenyl-2-yl)-3-difluoromethyl-1-methylpyrazol-4-ylcarboxamide,
N-(3′,4′,5′-trifluorobiphenyl-2-yl)-3-difluoromethyl-5-fluoro-1-methylpyrazol-4-ylcarboxamide,
N-(3′,4′,5′-trifluorobiphenyl-2-yl)-5-chloro-3-difluoromethyl-1-methylpyrazol-4-ylcarboxamide,
N-(3′,4′,5′-trifluorobiphenyl-2-yl)-3-(chlorodifluoromethyl)-1-methylpyrazol-4-ylcarboxamide,
N-(3′,4′,5′-trifluorobiphenyl-2-yl)-1-methyl-3-trifluoromethylpyrazol-4-ylcarboxamide,
N-(3′,4′,5′-trifluorobiphenyl-2-yl)-5-fluoro-1-methyl-3-trifluoromethylpyrazol-4-ylcarboxamide,
N-(3′,4′,5′-trifluorobiphenyl-2-yl)-5-chloro-1-methyl-3-trifluoromethylpyrazol-4-ylcarboxamide,
N-(2′,4′,5′-trifluorobiphenyl-2-yl)-1,3-dimethylpyrazol-4-ylcarboxamide,
N-(2′,4′,5′-trifluorobiphenyl-2-yl)-1,3-dimethyl-5-fluoropyrazol-4-ylcarboxamide,
N-(2′,4′,5′-trifluorobiphenyl-2-yl)-5-chloro-1,3-dimethylpyrazol-4-ylcarboxamide,
N-(2′,4′,5′-trifluorobiphenyl-2-yl)-3-fluoromethyl-1-methylpyrazol-4-ylcarboxamide,
N-(2′,4′,5′-trifluorobiphenyl-2-yl)-3-(chlorofluoromethyl)-1-methylpyrazol-4-ylcarboxamide,
N-(2′,4′,5′-trifluorobiphenyl-2-yl)-3-difluoromethyl-1-methylpyrazol-4-ylcarboxamide,
N-(2′,4′,5′-trifluorobiphenyl-2-yl)-3-difluoromethyl-5-fluoro-1-methylpyrazol-4-ylcarboxamide,
N-(2′,4′,5′-trifluorobiphenyl-2-yl)-5-chloro-3-difluoromethyl-1-methylpyrazol-4-ylcarboxamide,
N-(2′,4′,5′-trifluorobiphenyl-2-yl)-3-(chlorodifluoromethyl)-1-methylpyrazol-4-ylcarboxamide,
N-(2′,4′,5′-trifluorobiphenyl-2-yl)-1-methyl-3-trifluoromethylpyrazol-4-ylcarboxamide,
N-(2′,4′,5′-trifluorobiphenyl-2-yl)-5-fluoro-1-methyl-3-trifluoromethylpyrazol-4-ylcarboxamide,
N-(2′,4′,5′-trifluorobiphenyl-2-yl)-5-chloro-1-methyl-3-trifluoromethylpyrazol-4-ylcarboxamide,
N-(3′,4′-dichloro-3-fluorobiphenyl-2-yl)-1-methyl-3-trifluoromethyl-1H-pyrazole-4-carboxamide,
N-(3′,4′-dichloro-3-fluorobiphenyl-2-yl)-1-methyl-3-difluoromethyl-1H-pyrazole-4-carboxamide,
N-(3′,4′-difluoro-3-fluorobiphenyl-2-yl)-1-methyl-3-trifluoromethyl-1H-pyrazole-4-carboxamide,
N-(3′,4′-difluoro-3-fluorobiphenyl-2-yl)-1-methyl-3-difluoromethyl-1H-pyrazole-4-carboxamide,
N-(3′-chloro-4′-fluoro-3-fluorobiphenyl-2-yl)-1-methyl-3-difluoromethyl-1H-pyrazole-4-carboxamide,
N-(3′,4′-dichloro-4-fluorobiphenyl-2-yl)-1-methyl-3-trifluoromethyl-1H-pyrazole-4-carboxamide,
N-(3′,4′-difluoro-4-fluorobiphenyl-2-yl)-1-methyl-3-trifluoromethyl-1H-pyrazole-4-carboxamide,
N-(3′,4′-dichloro-4-fluorobiphenyl-2-yl)-1-methyl-3-difluoromethyl-1H-pyrazole-4-carboxamide,
N-(3′,4′-difluoro-4-fluorobiphenyl-2-yl)-1-methyl-3-difluoromethyl-1H-pyrazole-4-carboxamide,
N-(3′-chloro-4′-fluor-4-fluorobiphenyl-2-yl)-1-methyl-3-difluoromethyl-1H-pyrazole-4-carboxamide,
N-(3′,4′-dichloro-5-fluorobiphenyl-2-yl)-1-methyl-3-trifluoronnethyl-1H-pyrazole-4-carboxamide,
N-(3′,4′-difluoro-5-fluorobiphenyl-2-yl)-1-methyl-3-trifluoromethyl-1H-pyrazole-4-carboxamide,
N-(3′,4′-dichloro-5-fluorobiphenyl-2-yl)-1-methyl-3-difluoromethyl-1H-pyrazole-4-carboxamide,
N-(3′,4′-difluoro-5-fluorobiphenyl-2-yl)-1-methyl-3-difluoromethyl-1H-pyrazole-4-carboxamide,
N-(3′,4′-dichloro-5-fluorobiphenyl-2-yl)-1,3-dimethyl-1H-pyrazol-4-carboxamide,
N-(3′-chloro-4′-fluoro-5-fluorobiphenyl-2-yl)-1-methyl-3-difluoromethyl-1H-pyrazole-4-carboxamide,
N-(4′-fluoro-4-fluorobiphenyl-2-yl)-1-methyl-3-trifluoromethyl-1H-pyrazole-4-carboxamide,
N-(4′-fluoro-5-fluorobiphenyl-2-yl)-1-methyl-3-trifluoromethyl-1H-pyrazole-4-carboxamide,
N-(4′-chloro-5-fluorobiphenyl-2-yl)-1-methyl-3-trifluoromethyl-1H-pyrazole-4-carboxamide,
N-(4′-methyl-5-fluorobiphenyl-2-yl)-1-methyl-3-trifluoromethyl-1H-pyrazole-4-carboxamide,
N-(4′-fluoro-5-fluorobiphenyl-2-yl)-1,3-dimethyl-1H-pyrazole-4-carboxamide,
N-(4′-chloro-5-fluorobiphenyl-2-yl)-1,3-dimethyl-1H-pyrazole-4-carboxamide,
N-(4′-methyl-5-fluorobiphenyl-2-yl)-1,3-dimethyl-1H-pyrazole-4-carboxamide,
N-(4′-fluoro-6-fluorobiphenyl-2-yl)-1-methyl-3-trifluoromethyl-1H-pyrazole-4-carboxamide,
N-(4′-chloro-6-fluorobiphenyl-2-yl)-1-methyl-3-trifluoromethyl-1H-pyrazole-4-carboxamide,
N-[2-(1,1,2,3,3,3-hexafluoropropoxy)-phenyl]-3-difluoromethyl-1-methyl-1H-pyrazole-4-carboxamide,
N-[4′-(trifluoromethylthio)biphenyl-2-yl]-3-difluoromethyl-1-methyl-1H-pyrazole-4-carboxamide, and
N-[4′-(trifluoromethylthio)biphenyl-2-yl]-1-methyl-3-trifluoromethyl-1-methyl-1H-pyrazole-4-carboxamide.

Carbamate fungicides are fungicidally active compounds which comprise a carbamate group (NRR′—CO—OR″).

Carbamate fungicides and processes for their preparation are, in principle, known to the skilled worker and described for example in Farm Chemicals Handbook, Meister Publishing Company or in the Compendium of Pesticide Common Names, http://www.hclrss.demon.co.uk/, hereby fully incorporated herein by reference.

Preferred carbamate fungicides are those which are known under the common names benthiavalicarb, furophanate, iprovalicarb, propamocarb, thiophanate, thiophanate-methyl, thiophanate-ethyl, benomyl, carbendazim, cypendazol, debacarb and mecarbinzid. Among these, carbendazim, thiophanate, thiophanate-methyl and thiophanate-ethyl are especially preferred. In particular, thiophanate-methyl is used.

Dicarboximide fungicides are fungicidally active compounds which comprise an imide group of a dicarboxylic acid. Accordingly, these compounds comprise a cyclic structure having a —CO—NR—CO— group.

Dicarboximide fungicides and processes for their preparation are, in principle, known to the skilled worker and described for example in Farm Chemicals Handbook, Meister Publishing Company or in the Compendium of Pesticide Common Names, http://www.hclrss.demon.co.uk/, hereby fully incorporated herein by reference.

Preferred dicarboximides are those of the formula II

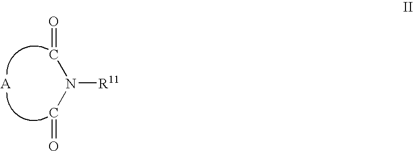

in which
A is —CR¹²R¹³—CR¹⁴R¹⁵—, —CR¹²R¹³—O—, —CR¹²R¹³—NR¹⁶— or —CR¹²═CR¹⁴—,
R¹¹ is C₁-C₈-alkylthio, C₁-C₈-haloalkylthio, C₁-C₈-alkylthio-C₁-C₄-alkyl, C₁-C₈-halo-alkylthio-C₁-C₄-alkyl, phenylthio, phenylthio-C₁-C₄-alkyl, phenyl, phenylamino, it being possible for phenyl in the four last-mentioned radicals to be partially or fully halogenated and/or to have attached to it 1 to 3 substituents which are selected among halogen, C₁-C₈-alkyl, C₁-C₈-alkoxy, phenyl and phenoxy, or R¹¹ is di(C₁-C₈-alkyl)phosphonate or di(C₁-C₈-alkyl)thiophosphonate;

R¹², R¹³, R¹⁴ and R¹⁵ independently of one another are hydrogen, halogen, C₁-C₈-alkyl, C₁-C₈-haloalkyl, C₁-C₈-alkoxy, C₁-C₈-alkylthio, C₁-C₈-haloalkoxy, C₁-C₈-haloalkylthio, C₁-C₈-alkoxy-C₁-C₈-alkyl, C₂-C₈-alkenyl, C₂-C₈-alkynyl, carboxyl (═COOH), C₁-C₈-alkyloxycarbonyl, C₁-C₈-alkylcarbonyl, C₁-C₈-alkylcarbonyloxy, phenyl which can be partially or fully halogenated and/or have attached to it 1 to 3 substituents which are selected among halogen, C₁-C₈-alkyl, C₁-C₈-alkoxy, phenyl, phenoxy, benzyl and benzyloxy,

where
R¹² and R¹⁴ together with the carbon atoms to which they are bonded can also form a 3- to 6-membered saturated or unsaturated aromatic or nonaromatic cycle which can be unsubstituted or substituted by 1 to 3 substituents which are selected among halogen, C₁-C₈-alkyl, C₁-C₈-alkoxy, phenyl, phenoxy, benzyl or benzoxy; and
R¹⁶ is hydrogen, C₁-C₄-alkyl, C₁-C₈-alkylcarbonyl, C₁-C₈-alkyloxycarbonyl or C₁-C₈-alkylaminocarbonyl or di(C₁-C₈-alkyl)aminocarbonyl.

Preferred dicarboximide fungicides are those which are known under the common names famoxadone, fluoroimide, chlozolinate, dichlozoline, iprodione, isovaledione, myclozolin, procymidone, vinclozolin, captafol, captan, ditalimfos, folpet and thiochlorfenphim. Especially preferred are iprodione, vinclozolin and procymidone. In particular, iprodione is used.

Azole fungicides, which are also referred to as conazole fungicides, are fungicidally active compounds which comprise an aromatic 5-membered nitrogen heterocycle and in particular an imidazole ring (“imidazole conazole”) or a triazole ring (“triazole conazole”).

Azole fungicides and processes for their preparation are, in principle, known to the skilled worker and described for example in Farm Chemicals Handbook, Meister Publishing Company or in the Compendium of Pesticide Common Names, http://www.hclrss.demon.co.uk/, hereby fully incorporated herein by reference.

Preferred azole fungicides are those which are known under the common names bitertanol, bromoconazole, cyproconazole, difenoconazole, dinitroconazole, epoxiconazole, fenbuconazole, fluquinconazole, flusilazol, hexaconazole, imazalil, metconazole, myclobutanil, paclobutrazol, penconazole, propiconazole, prochloraz, prothioconazole, tebuconazole, triadimefon, triadimenol, triflumizol and triticonazole. Especially preferred are difenoconazole, flusilazol, metconazole, paclobutrazol, prothioconazole and tebuconazole. More preferred are flusilazol, metconazole, prothioconazole and tebuconazole. Even more preferred are metconazole, prothioconazole and tebuconazole. In particular, metconazole is used.

Strobilurin fungicides are fungicidally active compounds which are derived from natural strobilurins, defense substances which are produced by fungi of the genus Strobilurus. As regards their structure, they comprise 1.) at least one functional group which is selected among enol ethers, oxime ethers and O-alkylhydroxylamines (group I) and 2.) at least one carboxyl derivative (group II). Preferred carboxyl derivatives are the following functional groups: ester, cyclic ester, amide, cyclic amide, hydroxamic acid and cyclic hydroxamic acid. Preferably, the group I radicals and the group II radicals are directly adjacent to one another, i.e. linked via a single bond.

Strobilurin fungicides are, in principle, known to the skilled worker and described for example in Farm Chemicals Handbook, Meister Publishing Company or in the Compendium of Pesticide Common Names, http://www.hclrss.demon.co.uk/, hereby fully incorporated herein by reference.

Preferred strobilurins are those of the formulae IIIA or IIIB

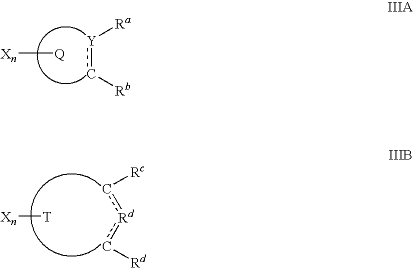

in which
[symbol] is a double bond or single bond;
R^{a} is —C[CO₂CH₃]═CHOCH₃, —C[CO₂CH₃]═NOCH₃, —C[CONHCH₃]═NOCH₃, —C[CO₂CH_{3]═CHCH}₃, —C[CO₂CH₃]═CHCH₂CH₃, —C[CO₂CH₃]═NOCH₃, —C[COCH₂CH₃]═NOCH₃, —N(OCH₃)—CO₂CH₃, —N(CH₃)—CO₂CH₃ or —N(CH₂CH₃)—CO₂CH₃;
R^{b} is an organic radical which is bonded directly or via an oxygen atom, a sulfur atom, an amino group or a C₁-C₈-alkylamino group; or
   together with a group X and the ring Q or T, to which they are bonded, an optionally substituted bicyclic, partially or fully unsaturated system which, in addition to carbon ring members, may comprise 1, 2 or 3 heteroatoms which are independently selected among oxygen, sulfur and nitrogen;
R^{c} is —OC[CO₂CH₃]═CHOCH₃, —OC[CO₂CH₃]═CHCH₃, —OC[CO₂CH₃]═CHCH₂CH₃, —SC[CO₂CH₃]═CHOCH₃, —SC[CO₂CH₃]═CHCH₃, —SC[CO₂CH₃]═CHCH₂CH₃, —N(CH₃)C[CO₂CH₃]═CHOCH₃, —N(CH₃)C[CO₂CH₃]═NOCH₃, —CH₂C[CO₂CH₃]═CHOCH₃, —CH₂C[CO₂CH₃]═NOCH₃ or —CH₂C[CONHCH₃]═NOCH₃;
R^{d} is oxygen, sulfur, ═CH— or ═N—;
n is 0, 1, 2 or 3, where, if n>1, the radicals X can be identical or different;
X is cyano, nitro, halogen, C₁-C₈-alkyl, C₁-C₈-haloalkyl, C₁-C₈-alkoxy, C₁-C₈-haloalkoxy or C₁-C₈-alkylthio, or
   if n>1, a C₃-C₅-alkylene, C₃-C₅-alkenylene, oxy-C₂-C₄-alkylene, oxy-C₁-C₃-alkylenoxy, oxy-C₂-C₄-alkenylene, oxy-C₂-C₄-alkenylenoxy or butadienediyl group which is bonded to two adjacent C atoms of the phenyl ring, it being possible for these chains, in turn, to have attached to them one to three radicals which are independently of one another selected among halogen, C₁-C₈-alkyl, C₁-C₈-haloalkyl, C₁-C₈-alkoxy, C₁-C₈-haloalkoxy and C₁-C₈-alkylthio;
Y is ═C— or —N—;
Q is phenyl, pyrrolyl, thienyl, furyl, pyrazolyl, imidazolyl, oxazolyl, isoxazolyl, thiazolyl, thiadiazolyl, triazolyl, pyridinyl, 2-pyridonyl, pyrimidinyl or triazinyl; and
T is phenyl, oxazolyl, thiazolyl, thiadiazolyl, oxadiazolyl, pyridinyl, pyrimidinyl or triazinyl.

In particular, the substituent R^{b} is a C₁-C₈-alkyl, C₂-C₈-alkenyl, C₂-C₈-alkynyl, aryl, hetaryl, aryl-C₁-C₈-alkyl, hetaryl-C₁-C₈-alkyl, aryl-C₂-C₈-alkenyl, hetaryl-C₂-C₈-alkenyl, aryl-C₂-C₈-alkynyl or hetaryl-C₂-C₈-alkynyl radical which is optionally interrupted by one or more groups which are selected among O, S, SO, SO₂, NR(R═H or C₁-C₈-alkyl), CO, COO, OCO, CONH, NHCO and NHCONH or a radical of the formulae defined hereinbelow CH₂ON═CR^{α}CR^{β} or CH₂ON═CR^{γ}CR^{δ}═NOR^{ε}. These radicals optionally also have one or more (preferably 1, 2 or 3) substituents which are independently of one another selected among C₁-C₈-alkyl, C₁-C₈-alkoxy, halogen, cyano, C₁-C₈-haloalkyl (in particular CF₃ and CHF₂), hetaryl and aryl. Hetaryl and aryl, in turn, can have 1, 2 or 3 substituents which are independently of one another selected among halogen, C₁-C₈-haloalkyl (in particular CF₃ and CHF₂), phenyl, CN, phenoxy, C₁-C₈-alkyl, C₁-C₈-alkoxy and C₁-C₈-haloalkoxy.

Such compounds are known and described for example in WO 97/10716 and in the literature cited therein, hereby fully incorporated herein by reference.

Preferred strobilurins are those of the formulae IIIA or IIIB in which R^{b} is aryloxy, hetaryloxy, aryloxymethylene, hetaryloxymethylene, arylethenylene or hetarylethenylene, these radicals optionally having 1, 2 or 3 substituents which are independently of one another selected among C₁-C₈-alkyl, halogen, CF₃, CHF₂, CN, C₁-C₈-alkoxy and phenyl which, in turn, can have 1, 2 or 3 substituents which are independently of one another selected among halogen, CF₃, CHF₂, phenyl, CN, phenoxy, C₁-C₈-alkyl, C₁-C₈-alkoxy and C₁-C₈-haloalkoxy;

### or R^{b} is CH₂ON═CR^{α}R^{β} or CH₂ON═CR^{γ}CR^{δ}═NOR^{ε},

where
R^{α} is C₁-C₈-alkyl;
R^{β} is phenyl, pyridyl or pyrimidyl, optionally having 1, 2 or 3 substituents which are independently of one another selected among C₁-C₈-alkyl, C₁-C₈-alkoxy, halogen, C₁-C₈-haloalkoxy, CF₃ and CHF₂;
R^{γ} is C₁-C₈-alkyl, C₁-C₈-alkoxy, halogen, C₁-C₈-haloalkyl or hydrogen;
R^{δ} is hydrogen, cyano, halogen, C₁-C₈-alkyl, C₁-C₈-alkoxy, C₁-C₈-alkylthio, C₁-C₈-alkylamino, di-C₁-C₈-alkylamino, C₂-C₈-alkenyl, C₂-C₈-alkenyloxy, C₂-C₈-alkenylthio, C₂-C₈-alkenylamino, N—C₂-C₈-alkenyl-N—C₁-C₈-alkylamino, C₂-C₈-alkynyl, C₂-C₈-alkynyloxy, C₂-C₈-alkynylthio, C₂-C₈-alkynylamino, N—C₂-C₈-alkynyl-N—C₁-C₈-alkylamino, it being possible for the hydrocarbon radicals of these groups to be partially or fully halogenated and/or to have attached to them 1, 2 or 3 radicals which are independently of one another selected among cyano, nitro, hydroxyl, C₁-C₈-alkoxy, C₁-C₈-haloalkoxy, C₁-C₈-alkoxycarbonyl, C₁-C₈-alkylthio, C₁-C₈-alkylamino, di-C₁-C₈-alkylamino, C₂-C₈-alkenyloxy, C₃-C₈-cycloalkyl, C₃-C₈-cycloalkyloxy, heterocyclyl, heterocyclyloxy, aryl, aryloxy, aryl-C₁-C₈-alkoxy, hetaryl, hetaryloxy and hetaryl-C₁-C₈-alkoxy, it being possible for the cyclic radicals, in turn, to be partially or fully halogenated and/or to have attached to them 1, 2 or 3 groups which are independently of one another selected among cyano, nitro, hydroxyl, C₁-C₈-alkyl, C₁-C₈-haloalkyl, C₃-C₈-cycloalkyl, C₁-C₈-alkoxy, C₁-C₈-haloalkoxy, C₁-C₈-alkoxycarbonyl, C₁-C₈-alkylthio, C₁-C₈-alkylamino, di-C₁-C₈-alkylamino, C₂-C₈-alkenyl and C₂-C₈-alkenyloxy; or
   is C₃-C₈-cycloalkyl, C₃-C₈-cycloalkyloxy, C₃-C₈-cycloalkylthio, C₃-C₈-cycloalkylamino, N—C₃-C₈-cycloalkyl-N—C₁-C₈-alkylamino, heterocyclyl, heterocyclyloxy, heterocyclylthio, heterocyclylamino, N-heterocyclyl-N—C₁-C₈-alkylamino, aryl, aryloxy, arylthio, arylamino, N-aryl-N—C₁-C₈-alkylamino, hetaryl, hetaryloxy, hetarylthio, hetarylamino or N-hetaryl-N—C₁-C₈-alkylamino, it being possible for the cyclic radicals to be partially or fully halogenated and/or to have attached to them 1, 2 or 3 groups which are independently of one another selected among cyano, nitro, hydroxyl, C₁-C₈-alkyl, C₁-C₈-haloalkyl, C₃-C₈-cycloalkyl, C₁-C₈-alkoxy, C₁-C₈-haloalkoxy, C₁-C₈-alkoxycarbonyl, C₁-C₈-alkylthio, C₁-C₈-alkylamino, di-C₁-C₈-alkylamino, C₂-C₈-alkenyl, C₂-C₈-alkenyloxy, benzyl, benzyloxy, aryl, aryloxy, hetaryl and hetaryloxy, it being possible for the aromatic radicals in turn to be partially or fully halogenated and/or to have attached to them 1, 2 or 3 of the following groups: cyano, C₁-C₈-alkyl, C₁-C₈-haloalkyl, C₁-C₈-alkoxy, nitro;
R⁶ is C₁-C₈-alkyl, C₂-C₈-alkenyl or C₂-C₈-alkynyl, it being possible for these groups to be partially or fully halogenated and/or to have attached to them 1, 2 or 3 of the following radicals: cyano, C₁-C₈-alkoxy, C₃-C₈-cycloalkyl.

Particularly preferred compounds of the formula IIIA or IIIB are those in which R^{b} has one of the following meanings:

a) phenyloxymethylene, pyridinyloxymethylene, pyrimidinyloxymethylene or pyrazolyloxymethylene, the aromatic radical optionally having 1, 2 or 3 substituents which are independently of one another selected among C₁-C₈-alkyl, halogen, CF₃, CHF₂, —C(CH₃)═NOCH₃ and phenyl which is optionally substituted by 1, 2 or 3 halogen atoms and/or C₁-C₈-alkyl groups;
b) phenoxy or pyrimidinyloxy which is optionally substituted by 1, 2 or 3 halogen atoms or by a phenoxy radical which optionally has a halogen or cyano substituent;
c) phenylethenylene or pyrazolylethenylene, the phenyl or pyrazolyl radical optionally having 1, 2 or 3 substituents which are independently of one another selected among halogen, CF₃, CHF₂ and phenyl;

### d) CH₂ON═CR^{α}R^{β}

in which
R^{α} is C₁-C₈-alkyl; and
R^{β} is phenyl which optionally has 1, 2 or 3 substituents which are independently of one another selected among C₁-C₈-alkyl, halogen, CF₃ and CHF₂, or is pyrimidinyl which is optionally substituted by 1 or 2 C₁-C₈-alkoxy radicals;
   e) CH₂ON═CR^{γ}CR^{δ}═NOR^{ε}, where
R^{γ} is C₁-C₈-alkyl, C₁-C₈-alkoxy or halogen;
R^{δ} is C₁-C₈-alkyl, cyano, halogen, C₁-C₈-alkoxy, C₁-C₈-alkenyl or phenyl which is optionally substituted by 1, 2 or 3 halogen atoms; and
R^{ε} is C₁-C₈-alkyl.

Especially preferred compounds of the formula IIIA are those in which Q is phenyl and n is O.

Particularly preferred strobilurins are those which are known under the common names azoxystrobin, dimoxystrobin, fluoxastrobin, kresoxim-methyl, methaminostrobin, orysastrobin, picoxystrobin, pyraclostrobin and trifloxystrobin. More preferred are pyraclostrobin, azoxystrobin and dimoxystrobin. Even more preferred are azoxystrobin and dimoxystrobin, in particular dimoxystrobin.

Morpholine fungicides are fungicidally active compounds which comprise a morpholine group

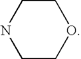

Morpholine fungicides and processes for their preparation are, in principle, known to the skilled worker and described for example in Farm Chemicals Handbook, Meister Publishing Company or in the Compendium of Pesticide Common Names, http://www.hclrss.demon.co.uk/, hereby fully incorporated herein by reference.

Preferred morpholine fungicides are those which are known under the common names aldimorph, benzamorf, carbamorph, dimethomorph, dodemorph, fenpropimorph, flumorph and tridemorph. Among these dimethomorph is particularly preferred. The growth regulators are preferably selected among

(a) acylcyclohexanediones of the formula (IV)

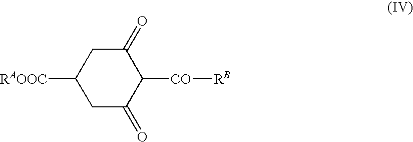

in which
R^{A} is H or C₁-C₁₀-alkyl and
R^{B} is C₁-C₁₀-alkyl or C₃-C₁₀-cycloalkyl
   or salts thereof;
   (b) quaternary ammonium compounds of the formula (V)

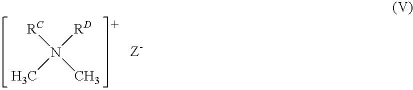

in which
R^{C} and R^{D} independently of one another are C₁-C₁₀-alkyl which is optionally substituted by at least one halogen atom, or a C₃-C₁₀-cycloalkyl; or
R^{C} and R^{D} together form a bridging unit —(CH₂)ₙ—, —(CH₂)₂—O—(CH₂)₂— or —(CH₂)—CH═CH—(CH₂)—NH—,
   in which n is 4 or 5, and
Z⁻ is a counter anion which is selected among halide ions, sulfate ions, C₁-C₁₀-alkylsulfonate ions, borate ions, carbonate ions and mixtures of these; and
   (c) ethephone (2-chloroethylphosphonic acid).

Sulfate ions are not only the pure sulfate anion SO₄²⁻, but also C₁-C₁₀-alkyl sulfate ions RO—S(O)₂—O⁻ in which R is C₁-C₁₀-alkyl, for example methyl sulfate, ethyl sulfate and the like. Preferably, it is the pure sulfate anion SO₄²⁻.

C₁-C₁₀-Alkylsulfonate ions are anions of the formula R—S(O)₂—O—, in which R is C₁-C₁₀-alkyl, for example methylsulfonate, ethylsulfonate and the like.

The borate anions are preferably those of the formula VI

1/m.[MₓB_{y}O_{z}(A)ᵥ]^{m-}.w(H₂O) (VI)

in which
M is a cation of an agriculturally tolerated metal, a proton or ammonium;
A is a chelating or complexing group which is associated with at least one boron atom or a cation M;
x is a number from 0 to 10;
y is a number from 1 to 48;
z is a number from 0 to 48;
v is a number from 0 to 24;
m is a number from 1 to 6;
w is a number from 0 to 24.

M is preferably a cation of the metal selected among sodium, potassium, magnesium, calcium, zinc, manganese and copper, a proton or ammonium.

A is preferably selected among hydroxycarboxylic acid, carboxylic acid, alcohols, glycols, amino alcohols, sugars and the like.

Examples of suitable hydroxycarboxylic acids are glycolic acid, lactic acid, mandelic acid, malic acid, tartaric acid, citric acid, other fruit acids and also hydroxy fatty acids such as ricinoleic acid.

Suitable carboxylic acids are monocarboxylic acids such as formic acid, acetic acid, propionic acid, valeric acid, isovaleric acid, caproic acid, enanthic acid, caprylic acid and other fatty acids, and dicarboxylic acids such as oxalic acid, malonic acid, succinic acid, adipic acid and the like.

Examples of suitable alcohols are C₁-C₈-alcohols such as methanol, ethanol, propanol, isopropanol, n-butanol, sec-butanol, isobutanol, tert-butanol, pentyl alcohols such as pentanol and amyl alcohol, hexyl alcohols such as hexanol, heptyl alcohols such as heptanol and octyl alcohols such as octanol and 2-ethylhexanol.

Examples of suitable glycols are C₂-C₁₀-diols such as glycol, diethylene glycol, triethylene glycol and the like.

Examples of suitable amino alcohols are ethanolamine, diethanolamine, triethanolamine and the like.

Examples of suitable sugars are the pentoses and hexoses, such as fructose, glucose, mannose and the like, and also the disaccharides such as sucrose.

x preferably is 0, especially when M does not have one of the abovementioned preferred meanings.

y preferably is a number from 2 to 20, particularly preferably from 2 to 10, more preferably from 3 to 10, even more preferably from 3 to 7 and in particular from 3 to 5. Specifically, y represents 5.

z is preferably a number from 6 to 10, particularly preferably from 6 to 8 and in particular 8.

v is preferably 0.

w is preferably a number from 2 to 10, particularly preferably from 2 to 8 and in particular 2 or 3.

m is preferably 1 or 2 and in particular 1.

Preferred are borates of the formula (VI) in which x is zero; or M is a cation of a metal selected among sodium, potassium, magnesium, calcium, zinc, manganese and copper, a proton or ammonium; and/or y is a number from 2 to 20, preferably 2 to 10, particularly preferably 3 to 10, more preferably 3 to 7, in particular 3 to 5; and/or z is a number from 6 to 10, in particular 6 to 8; and/or v is zero; and/or m is 1 or 2; and/or w is a number from 0 to 24.

Especially preferred are borates of the formula (VI) in which y is a number from 3 to 7, in particular 3 to 5; z is a number from 6 to 10, in particular 6 to 8; v is zero; and w is a number from 2 to 10, in particular 2 to 8.

Very especially preferred are borates of the formula (VI) in which y=5; z=8; v=0; m=1; w=2 to 3 (pentaborate).

If required, the charge in the borates is counterbalanced via the cation M.

The borates may comprise water constituents, for example as water of crystallization in free or coordinated form or as bound water in the form of borone-bound hydroxyl groups.

Suitable and preferred borates and processes for their preparation are known per se and described, for example, in WO 02/083732 and in the literature cited therein, hereby fully incorporated herein by reference. Other suitable borates are, for example, described in WO 99/09832, hereby fully incorporated herein by reference.

The compounds of the formulae (IV) and (V) are known (see, for example, EP-A-123001, EP-A-126713, W. Rademacher, “Growth Retardants: Effects on Gibberellin Biosynthesis and Other Metabolic Pathways”, Annu. Rev. Plant. Mol. Biol. 2000, 51, 501-531).

The compounds of the formula (IV) can exist both in the trione form (triketo form) IV.a and in the tautomeric keto-enol forms IV.b and IV.c, respectively:

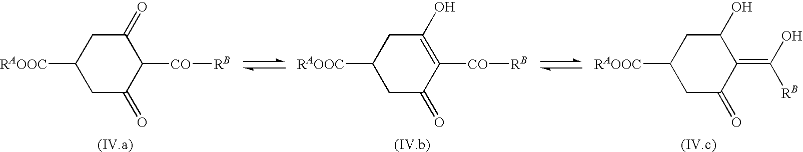

In the compounds of the formula IV, R^{A} is preferably H or C₁-C₄-alkyl.

R^{B} is preferably C₁-C₄-alkyl or C₃-C₆-cycloalkyl and in particular ethyl or cyclopropyl.

The salts of the acylcyclohexanedione compounds IV where R^{A}≠H are the salts of mono-anions, while in the case of R^{A}=H they may take the form of the mono- and of the di-anions of these compounds. The mono-anions may be present both as carboxylate anions IV.d and as enolate anions IV.e and IV.f, respectively:

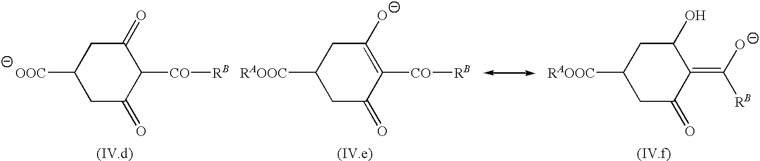

The carboxylate and enolate groups are present correspondingly alongside one another in the di-anions.

Preferred cations in the salts of the compounds of the formula IV are the ions of the alkali metals, preferably of lithium, sodium and potassium, of the alkaline earth metals, preferably of calcium and magnesium, and of the transition metals, preferably of manganese, copper, zinc and iron, furthermore ammonium (NH₄⁺) and substituted ammonium in which from one to four hydrogen atoms are replaced by C₁-C₄-alkyl, hydroxy-C₁-C₄-alkyl, C₁-C₄-alkoxy-C₁-C₄-alkyl, hydroxy-C₁-C₄-alkoxy-C₁-C₄-alkyl, phenyl or benzyl, preferably ammonium, methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxyeth-1-oxy)eth-1-ylammonium, di(2-hydroxyeth-1-yl)ammonium, benzyltrimethylammonium, benzyltriethylammonium, furthermore phosphonium ions, sulfonium ions, preferably tri(C₁-C₄-alkyl)sulfonium such as trimethylsulfonium, and sulfoxonium ions, preferably tri(C₁-C₄-alkyl)sulfoxonium. Preferred cations are furthermore chlormequat [(2-chloroethyl)trimethylammonium], mepiquat (N,N-dimethylpiperidinium) and N,N-dimethylmorpholinium. Particularly preferred cations are the alkali metal cations, the alkaline earth metal cations and the ammonium cation (NH₄⁺). In particular, it is the calcium salt.

In the context of the present invention, the term “compounds of the formula IV”, “acylcyclohexanediones of the formula IV” or “growth regulators of the formula IV” refer both to the neutral compounds IV and to their salts.

Compounds IV which are particularly preferably used in accordance with the invention are prohexadione (R^{A}=H, R^{B}=ethyl), prohexadione calcium (calcium salt of prohexadione), trinexapac (R^{A}=H, R^{B}=cyclopropyl) and trinexapac-ethyl (R^{A}=ethyl, R^{B}=cyclopropyl).

In compounds of the formula (V), one of the radicals R^{C} or R^{D} is preferably C₁-C₁₀-alkyl, while the other radical is C₁-C₁₀-alkyl which is substituted by a halogen atom, preferably by a chlorine atom. R^{C} is particularly preferably methyl and R^{D} is particularly preferably 2-chloroethyl.

In an alternatively preferred embodiment, R^{C} and R^{D} together form a bridging unit —(CH₂)₅—.

In a preferred embodiment of the invention, the anions Z⁻ in compounds V are selected among halide ions, sulfate ions and carbonate ions.

In an alternatively preferred embodiment of the invention, the anions Z⁻ in compounds V are selected among halide ions, especially chloride, borates, especially pentaborate, and mixtures of these.

Particularly preferably, Z⁻ is a halide anion and in particular chloride.

In particular, the quaternary ammonium compounds of the formula (V) are the salt of chlormequat (salt of 2-chloroethyltrimethylammonium), in particular chlormequatchloride (2-chloroethyltrimethylammonium chloride), or the salt of mepiquat (salt of 1,1-dimethylpiperidinium), in particular mepiquat-chloride (1,1-dimethylpiperidinium chloride).

Moreover, mixtures of the above-described growth regulators (IV), (V) and/or ethephone may also be employed.

Specifically, the growth regulators used are compounds (V).

In accordance with the invention, it is also possible to employ two or more of the abovementioned fungicides which are selected from the same class or from different classes of fungicides. The combined application (also referred to as combination of two or more fungicides in the context of the present invention) comprises both the use of a mixture of different fungicides and their separate use, it being possible in this case for the fungicides to be used simultaneously or else in succession, i.e. in an interval of, for example, a few seconds to several months.

The fungicides to be employed in accordance with the invention are preferably selected among aryl- and/or heterocyclylamides, strobilurins and azoles. As regards suitable and preferred representatives of these classes of fungicide, reference is made to what has been said above. Also preferred is the combined use of at least two representatives of these classes of fungicides. Specifically, at least one aryl- or heterocyclylamide is used in combination with at least one azole.

In a preferred embodiment of the invention, at least one aryl- and/or heterocyclylamide is used as fungicide. As regards suitable and preferred amides, reference is made to what has been said above. In particular, the amide fungicide used is boscalid.

In an alternatively preferred embodiment of the invention, at least one azole is used as fungicide. As regards suitable and preferred azoles, reference is made to what has been said above. It is preferred to use metconazole, prothioconazole or tebuconazole or their combination as azole fungicide. In particular, the azole fungicide used is metconazole.

In an alternatively preferred embodiment of the invention, at least one strobilurin is used as fungicide. As regards suitable and preferred strobilurins, reference is made to what has been said above. It is preferred to use azoxystrobin or dimoxystrobin or their combination as strobilurin fungicide. In particular, the strobilurin fungicide used is dimoxystrobin.

In an alternatively preferred embodiment of the invention, at least one aryl- or heterocyclylamide fungicide is used in combination with at least one azole fungicide. The preferred amide fungicide here is boscalid. The preferred azole fungicide is metconazole.

In an alternatively preferred embodiment of the invention, at least one aryl- or heterocyclylamide fungicide is used in combination with at least one strobilurin fungicide. The preferred amide fungicide here is boscalid. The preferred strobilurin fungicide is dimoxystrobin.

Particularly preferably, at least one aryl- or heterocyclylamide is used as fungicide, especially boscalid optionally in combination with at least one azole fungicide, especially with metconazole, or optionally in combination with at least one strobilurin fungicide, especially with dimoxystrobin, or particularly preferably at least one azole fungicide is used, especially metconazole. In particular at least one aryl- or heterocyclylamide is used as fungicide, especially boscalid, in combination with at least one azole fungicide, especially with metconazole.

If the at least one fungicide is employed in combination with at least one growth regulator, the weight ratio of fungicide to growth regulator is preferably 15:1000 to 1000:15, particularly preferably 3:50 to 25:7 and in particular 6:50 to 15:7.

The use according to the invention is generally effected in such a way that the oil crop or plant parts thereof or the seed of the oil crops are treated with these compounds. The treatment of the oil crops or of the seed is preferably effected in such a way that the oil crop or plant parts thereof or the seed are brought into contact with at least one of the fungicides employed in accordance with the invention and optionally with at least one growth regulator. To this end, at least one fungicide is applied to the plant or to plant parts thereof or to the seed. If a plurality of fungicides used in accordance with the invention are combined, they can be applied as a mixture or separately. In the case of separate application, the application of the individual active substances can be effected simultaneously or split within the context of a series of treatments; in the case of successive application, they can be applied at intervals of from a few seconds or a few minutes to several weeks or even a few months, for example up to 10 months. It is also possible repeatedly to apply a single active substance, for example at an interval between the individual applications of from a few seconds or a few minutes to several weeks or even a few months, for example up to 10 months. The same applies analogously to the optional treatment with at least one growth regulator, i.e. the at least one fungicide and the at least one growth regulator can be applied as a mixture or separately and, in the latter case, simultaneously or successively. In the case of successive application of the active substances, the latter may also be applied at different developmental stages of the plants. Thus, for example, one active substance may be applied to the seed from which the plant is to grow, while another, or else the same, active substance is applied to the plant or plant parts thereof at the developmental stage after emergence.

Naturally, the oil crops or parts thereof to be treated are live plants, or plant parts of live plants.

The application timing, the number of applications and the application rates applied in each case are to be adapted to the prevailing conditions and must be decided by the skilled worker for each individual case. Apart from the active substances used in each case, a differentiation must be made in particular as to whether intact plants are to be treated under field conditions or whether seed is to be treated.

If a plant or a plant part is treated, the treatment is preferably effected during growth stage 1 to 6, particularly preferably 2 to 6, more preferably 3 to 6 and in particular 3 to 5 (in accordance with BBCH Makrostadien; Biologische Bundesanstalt kir Land- und Forstwirtschaft [BBCH Macrostages; German Federal Biological Research Center for Agriculture and Forestry]; see www.bba.de/veroeff/bbch/bbch.htm).

In the case of the most preferred fungicides employed in accordance with the invention, which is the at least one aryl- or heterocyclylamide, especially boscalid, in combination with the at least one azole fungicide, especially metconazole, it is preferred to treat the plant or plant parts thereof with the at least one azole once or more than once before anthesis, preferably in the autumn and/or in the spring, especially preferably in the autumn and in the spring, and with the at least one aryl- or heterocyclylamide during anthesis.

Autumn and spring are relative concepts which depend on the hemisphere of the earth and on the respective vegetation zone and plant and which, for the purposes of the present invention, refer to those developmental phases of the plant in which the latter would be in central Europe during these seasons. Generally, autumn is the season in which the oil crop will be in growth stage 01 to 39, and spring before anthesis is the season in which the oil crop will be in growth stage 07 to 49 (according to extended BBCH scale; Biologische Bundesanstalt für Land- und Forstwirtschaft [Federal Biological Research Center for Agriculture and Forestry]; see www.bba.de/veroeff/bbch/bbch.htm). The overlap of the growth phases will depend on the weather in the respective year and on the individual plant species.

It is especially preferred to treat the oil crop or plant parts thereof with the at least one azole once or more than once, preferably once or twice, when the plant is in growth stage 01 to 29 and then again once or more than once, preferably once or twice, when the plant is in growth stage 30 to 39; thereafter, the oil crop or plant parts thereof are treated with the at least one aryl- or heterocyclylamide once or more than once, preferably once or twice, when the plant is in growth stage 50 to 69.

The active substances, as such or in the form of their formulations or in the form of the use forms prepared therefrom, can be applied by injecting, spraying, atomizing, dusting, scattering, pouring or dressing. The use forms depend entirely on the intended use, in particular on the plant species and variety and/or on the plant part, and the developmental stage of the plant to which they are to be applied; in any case, they should ensure as fine as possible a distribution of the active substances employed in accordance with the invention and also of the auxiliaries.

The fungicides used in accordance with the invention and the growth regulators which are optionally employed are typically employed in the form of formulations as are customary in the field of crop protection and the protection of stored products.

Examples of customary formulations are solutions, emulsions, suspensions, dispersions, pastes, dusts, materials for spreading, powders and granules.

The formulations are prepared in the known manner, for example by diluting the active substance with solvents and/or carriers, if desired using emulsifiers and dispersants.

Suitable solvents/auxiliaries are mainly:
Water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, dimethyl fatty amides, fatty acids and fatty acid esters. In principle, it is also possible to use solvent mixtures.
Carriers such as natural minerals (for example kaolins, clays, talc, chalk) and ground synthetic minerals (for example highly disperse silica, silicates).
Surface-active substances, such as alkali metal, alkaline earth metal, ammonium salts of aromatic sulfonic acids, for example lignosulfonic acid, phenolsulfonic acid, naphthalenesulfonic acid and dibutylnaphthalenesulfonic acid and of fatty acids, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol or nonylphenol, alkylphenyl polyglycol ether, tributylphenyl polyglycol ether, tristearylphenyl polyglycol ether, alkylaryl polyether alcohols, isotridecyl alcohol, alcohol and fatty alcohol/ethylene oxide condensates, ethoxylated castor oil, polyoxyethylene or polyoxypropylene alkyl ethers, ethoxylated polyoxypropylene, lauryl alcohol polyglycol ether acetate, sorbitol esters, lignin-sulfite waste liquors, methylcellulose or siloxanes. Examples of suitable siloxanes are polyether/polymethylsiloxane copolymers, which are also referred to as “spreaders” or “penetrants”.

Inert formulation auxiliaries, in particular for the preparation of directly sprayable solutions, emulsions, pastes or oil dispersions, which are suitable are essentially: mineral oil fractions of medium to high boiling point, such as kerosene or diesel oil, furthermore coal tar oils, and oils of vegetable or animal origin, aliphatic, cyclic and aromatic hydrocarbons, for example toluene, xylenes, paraffins, tetrahydronaphthalene, alkylated naphthalenes or their derivatives, alcohols such as methanol, ethanol, propanol, butanol and cyclohexanol, ketones such as cyclohexanone and isophorone, strongly polar solvents, for example dimethyl sulfoxide, N-methylpyrrolidone or water.

Powders, materials for spreading and dusts can be prepared by mixing or concomitantly grinding the active substances together with a solid carrier.

Granules, for example coated granules, impregnated granules and homogeneous granules, can be prepared by binding the active substances to solid carriers.

Examples of solid carriers are mineral earths such as silica gels, silicates, talc, kaolin, Attaclay, limestone, lime, chalk, bole, loess, clay, dolomite, diatomaceous earth, calcium sulfate, magnesium sulfate, magnesium oxide, ground synthetic materials, fertilizers such as, for example, ammonium sulfate, ammonium phosphate, ammonium nitrate, ureas and products of vegetable origin, such as cereal meal, tree bark meal, wood meal and nutshell meal, cellulose powders and other solid carriers.

In general, the formulations comprise the fungicides employed in accordance with the invention in a total amount of from 0.01 to 95% by weight, preferably of from 0.1 to 90% by weight, based on the total weight of the formulation.

Products (formulations) for dilution in water are, for example, water-soluble concentrates (SL), dispersible concentrates (DC), emulsifiable concentrates (EC), emulsions (EW, EO), suspensions (SC, OD, SE), water-dispersible and water-soluble granules (WG, SG) and water-dispersible and water-soluble powders (WP, SP). Products (formulations) for the direct application are, for example, dusts (DP), granules (GR, FG, GG, MG) and ULV solutions (UL).

Aqueous use forms can be prepared from stock formulations, such as concentrated solutions, emulsion concentrates, suspensions, pastes, wettable powders (sprayable powders, oil dispersions) or water-dispersible granules by addition of water and applied for example by spraying.

To prepare emulsions, pastes or oil dispersions, the fungicides employed in accordance with the invention, as such or dissolved in an oil or solvent, can be homogenized in water by means of wetters, stickers, dispersants or emulsifiers. However, it is also possible to prepare concentrates which consist of the active substance, wetters, stickers, dispersants or emulsifiers and, if appropriate, solvent or oil, and such concentrates are suitable for dilution with water. Naturally, the use forms will comprise the auxiliaries used in the stock formulations.

The active substance concentrations in preparations which are diluted with water can vary within substantial ranges. They are in general between 0.0001 and 10% by weight, preferably between 0.01 and 1% by weight.

Various types of oils, and wetters, safeners, adjuvants, other fungicides, insecticides, herbicides, bactericides or else foliar fertilizers comprising, for example, trace elements and/or oligoelements, can be added to the active substances, optionally also immediately before application (tank mix). These agents can also be applied separately to the fungicides employed in accordance with the invention, it being possible to carry out the separate application before, simultaneously with, or after the application of the fungicides. These agents can be admixed to the fungicides employed in accordance with the invention in a weight ratio of 1:200 to 200:1, preferably 1:100 to 100:1.

The combined use of the fungicides employed in accordance with the invention with further active substances conventionally used in crop protection, for example with other fungicides, can be effected by employing a mixture of these active substances (for example a joint formulation or tank mix), or else by applying the individual active substances separately, simultaneously or in succession.

When the fungicides used in accordance with the invention are employed in combination with at least one of the abovementioned agents, their use in combination with at least one fungicide other than the above and/or at least one insecticide is particularly suitable.

The following list of fungicides with which the fungicides employed in accordance with the invention can be used jointly is intended to illustrate the possible combinations, but not to impose any limitation:
acylalanines such as benalaxyl, metalaxyl, ofurace, oxadixyl,
amine derivatives such as aldimorph, dodine, dodemorph, fenpropimorph, fenpropidin, guazatine, iminoctadine, spiroxamin, tridemorph,
anilinopyrimidines such as pyrimethanil, mepanipyrim or cyprodinyl,
antibiotics such as cycloheximide, griseofulvin, kasugamycin, natamycin, polyoxin or streptomycin,
dithiocarbamates such as ferbam, nabam, maneb, mancozeb, metam, metiram, propineb, polycarbamate, thiram, ziram, zineb,
heterocyclic compounds such as anilazin, cyazofamide, dazomet, dithianone, fenamidon, fenarimol, fuberidazol, isoprothiolan, nuarimol, probenazol, proquinazide, pyrifenox, pyroquilon, quinoxyfen, silthiofam, thiabendazol, tricyclazol, triforine,
copper fungicides such as Bordeaux mixture, copper acetate, copper oxychloride, basic copper sulfate,
nitrophenyl derivatives such as binapacryl, dinocap, dinobuton, nitrophthalisopropyl,
phenylpyrroles such as fenpiclonil or fludioxonil,
sulfur,
other fungicides such as acibenzolar-S-methyl, carpropamid, chlorothalonil, cyflufenamid, cymoxanil, diclomezin, diclocymet, diethofencarb, edifenphos, ethaboxam, fenhexamid, fentin acetate, fenoxanil, ferimzone, fluazinam, fosetyl, fosetyl-aluminum, hexachlorobenzene, metrafenon, pencycuron, phthalide, toloclofos-methyl, quintozene, zoxamide,
cinnamamides and analogs such as flumetover or flumorph.

The fungicides employed in accordance with the invention and the growth regulators which are optionally employed are preferably applied to the oil crop plant or parts thereof. Naturally, the treatment will be carried out on a live plant. It is preferred to apply to the aerial part of the plant.

However, in the case of some fungicides, seed treatment is also suitable.

In an embodiment which is preferred for field applications, i.e. the application to live plants or plant parts thereof, the fungicides employed in accordance with the invention, and the growth regulators which are optionally employed are used in the form of an aqueous spray mixture. The application is preferably effected by spraying. Here, either all of the aerial part of the plant or only individual plant parts, such as flowers, fruits, leaves or individual shoots, are treated. The choice of the individual plant parts which are to be treated depends on the plant species and its developmental stage. It is preferred to treat all of the aerial part of the plant.

The fungicides employed in accordance with the invention are preferably applied 1 to 5 times, especially preferably 1 to 3 times and in particular once or twice per season. If the treatment is carried out repeatedly, at least the second, third, etc. treatment will, as a rule, take the form of a field application. As regards the preferred route and frequency of application in the preferred use of at least one aryl- or heterocyclylamide in combination with at least one azole, reference is made to what has been said above.

In the case of seed, the fungicides employed in accordance with the invention are used in a formulation conventionally used for this type of application.

For the treatment of seeds, it is possible to employ, in principle, all customary seed treatment, or seed dressing, methods, such as, for example, the dry seed treatment, solvent-based liquid treatment, wet seed treatment, slurry treatment or encrusting. Specifically, a procedure is followed in the treatment in which the seed is mixed, in a suitable device, for example a mixing device for solid or solid/liquid mixing partners, with the desired amount of seed-dressing product formulation either as such or after previous dilution with water until the product is uniformly distributed in the seed. Optionally, this is followed by a drying operation.

In the case of field application, the fungicides employed in accordance with the invention are generally employed in an amount of from 5 to 3000 g individual active substance per ha per season, preferably 10 to 1000, particularly preferably 50 to 500 g of individual active substance per ha per season.

In the case of application to seed, the fungicides employed according to the invention are generally employed in an amount of from 0.01 g to 500 g, preferably 0.5 g to 200 g, of individual active substance per kg seed.

In the case of field application, the growth regulators which are optionally employed are employed in an amount of from 10 to 1500 g of individual active substance per ha per season, preferably 25 to 650, particularly preferably 70 to 450 g of individual active substance per ha per season.

The growth regulators which are optionally employed are preferably applied 1 to 4 times, particularly preferably 1 to 3 times and in particular once or twice per season.

A further subject matter of the present invention is a method of increasing the quality and optionally the quantity of oil crop products, comprising the treatment of an oil crop or of plant parts thereof, or its seed, with at least one of the abovementioned fungicides, optionally in combination with at least one growth regulator, harvesting the seeds of the oil crop plant at a point in time when their water content is no more than 15% by weight based on the total seed weight, and obtaining the oil crop products.

Increasing the quality and optionally the quantity of oil crop products, is as defined above.

As regards suitable and preferred oil crops, oil crop products and fungicides, and the amounts and type of the application, reference is made to what has been said above.

The treatment of the oil crop or plant parts thereof during growth phase 1 to 6, particularly preferably 2 to 6, more preferably 3 to 6 and in particular 3 to 5 (in accordance with BBCH Makrostadien; Biologische Bundesanstalt für Land- and Forstwirtschaft [BBCH Macrostages; German Federal Biological Research Center for Agriculture and Forestry]; see www.bba.de/veroeff/bbch/bbch.htm) is preferred. In this context, the oil crop is preferably treated at least to some extent during the flowering phase, i.e. at least one fungicide is applied during the flowering phase and optionally the same fungicide or a different fungicide is employed during a different vegetation period. If a plurality of fungicides to be employed in accordance with the invention are combined, it is preferred to employ one fungicide during the flowering phase and the other fungicide(s) before the flowering phase, for example in spring and/or in the autumn. If amide fungicides are combined with azole fungicides, it is preferred to apply the amide fungicide(s) in the flowering phase and the azole fungicide(s) at an earlier point in time, for example in spring and/or in the autumn. As regards further details, reference is made to what has been said above.

Harvesting takes place when the water content of the seeds is no more than 15% by weight, for example 6 to 15% by weight, particularly preferably no more than 14% by weight, for example 14% by weight, in particular no more than 12% by weight, for example 6 to 12% by weight, and specifically no more than 9% by weight, for example 6 to 9% by weight, based on the total seed weight. Here, the optimal water content depends on the oil crop in question. Thus, in soybeans and maize, it is relatively close to the upper limit, for example at no more than 15% by weight, for example 10 to 15% by weight, and specifically at no more than 14% by weight, for example at 10-14% by weight, in the case of sunflower in the middle range, for example at no more than 13% by weight, for example 9 to 13% by weight and specifically at no more than 12% by weight, for example at 9 to 12% by weight, in the case of oilseed rape in the lower range, for example at no more than 11% by weight, for example 7 to 11% by weight and specifically no more than 9% by weight, for example at 7 to 9% by weight, and in the case of flax in an even lower range, for example at no more than 9% by weight, for example 6 to 9% by weight and specifically no more than 7% by weight, for example 6 to 7% by weight.

The water content can be determined using conventional analytical methods, for example by determining the weight loss on drying under defined conditions (for example 100° C. over a defined period) or via the determination of the electrical conductivity under defined conditions (especially a temperature), for example using a cereal moisture meter Pfeuffer HE Lite from Pfeuffer GmbH, Germany.

Obtaining oil from the oil-yielding parts of the plant, which are the seeds, fruits, and/or nuts of the oil crop, is accomplished in the manner conventionally used for the plant or plant product in question, for example by pressing and/or by extracting. The skilled worker is sufficiently familiar with the pre- or aftertreatment measures required in each case for the individual plants or their plant products.

Obtaining the oil by pressing generates, as residue, what is known as the presscake which, in turn, can be reused, for example, as feed or combustible.

The method according to the invention preferably leads to a reduction of the phosphorus content of the products of the treated plants, in particular of the oil obtained from the oil crops and/or its reaction products, for example its C₁-C₄-alkyl esters.

Alternatively, or additionally, the method according to the invention leads to a reduction of the alkali and/or alkaline earth metal content, especially the alkaline earth metal content and specifically the calcium and magnesium content of the products of the treated plants, in particular of the oil obtained from the oil crops and/or its reaction products, for example its C₁-C₄-alkyl esters.

Alternatively, or additionally, the method according to the invention leads to a reduction of the acid content (measured as the acid number) of the products of the treated plants, in particular of the oil obtained from the oil crops and optionally its reaction products, for example its C₁-C₄-alkyl esters.

Alternatively, or additionally, the method according to the invention leads to a reduction of the iodine number of the products of the treated plants, in particular of the oil obtained from the oil crops and/or its reaction products, for example its C₁-C₄-alkyl esters.

Alternatively, or additionally, the method according to the invention leads to an increase in the oxidation stability of the products of the treated plants, in particular of the oil obtained from the oil crops and optionally its reaction products, for example its C₁-C₄-alkyl esters.

Alternatively, or additionally, the method according to the invention leads to a reduction of the overall contamination of the products of the treated plants, in particular of the oil obtained from the oil crops and optionally its reaction products, for example its C₁-C₄-alkyl esters.

Alternatively, or additionally, the method according to the invention leads to a reduction of the kinematic viscosity of the products of the treated plants, in particular of the oil obtained from the oil crops and optionally its reaction products, for example its C₁-C₄-alkyl esters.

Alternatively, or additionally, the method according to the invention leads to a reduction of the sulfur content of the products of the treated plants, in particular of the oil obtained from the oil crops and optionally its reaction products, for example its C₁-C₄-alkyl esters.

Alternatively, or additionally, the method according to the invention leads to an increase of the flashpoint of the products of the treated plants, in particular of the oil obtained from the oil crops and optionally its reaction products, for example its C₁-C₄-alkyl esters.

Alternatively, or additionally, the method according to the invention leads to an increase of the calorific value of the products of the treated plants, in particular of the oil obtained from the oil crops and optionally its reaction products, for example its C₁-C₄-alkyl esters.

Alternatively, or additionally, the method according to the invention leads to a reduction of the carbon residue of the products of the treated plants, in particular of the oil obtained from the oil crops and optionally its reaction products, for example its C₁-C₄-alkyl esters.

Alternatively, or additionally, the method according to the invention leads to an increase of the cetane number of the products of the treated plants, in particular of the oil obtained from the oil crops and optionally its reaction products, for example its C₁-C₄-alkyl esters.

Alternatively, or additionally, the method according to the invention leads to a reduction of the nitrogen content of the products of the treated plants, in particular of the oil obtained from the oil crops and optionally its reaction products, for example its C₁-C₄-alkyl esters.

Alternatively, or additionally, the method according to the invention leads to a reduction of the chlorine content of the products of the treated plants, in particular of the oil obtained from the oil crops and optionally its reaction products, for example its C₁-C₄-alkyl esters.

Alternatively, or additionally, the method according to the invention leads to a reduction of the tin, zinc, silicon and/or boron content of the products of the treated plants, in particular of the oil obtained from the oil crops and optionally its reaction products, for example its C₁-C₄-alkyl esters.

The method according to the invention particularly preferably leads to an improvement of the properties listed under (i) to (xi), more preferably to an improvement of the properties listed under (i) to (viii) and in particular to an improvement of the properties listed under (i) to (vii), of the products of the treated plants, in particular of the oil obtained from the oil crops and optionally of its reaction products, for example its C₁-C₄-alkyl esters.

The method according to the invention especially preferably leads to a reduction of the phosphorus content and/or the alkali metal and/or alkaline earth metal content and/or the acid content, in particular to a reduction of the phosphorus content and/or the acid content of the products of the treated plants, in particular of the oil obtained from the oil crops and/or its reaction products, for example its C₁-C₄-alkyl esters. Accordingly, the process according to the invention is particularly preferably used for producing oil crop products, in particular vegetable oil and/or its reaction products, for example its C₁-C₄-alkyl esters, with a reduced phosphorus content and/or alkali metal and/or alkaline earth metal content and/or acid content and in particular with a reduced phosphorus content and/or acid content.

The acid content of the oil crop products, especially of the oil and optionally its reaction products, can be determined for example as specified in DIN EN 14104 (as acid number). The oxidation stability can be measured as specified in DIN EN 14112. The determination of the phosphorus content can be effected as specified in DIN EN 14107, and that of the alkali metal (especially. Na and K) and alkaline earth metal (calcium and magnesium) content as specified in DIN EN 14538. The determination of the iodine number can be effected as specified in EN 14111. The overall contamination can be measured for example as specified in EN 12662. The kinematic viscosity can be measured for example as specified in EN ISO 3104. The flashpoint can be measured for example as specified in EN ISO 2719, the net calorific value as specified in DIN 51900-1 and -3, the Conradson carbon residue as specified in EN ISO 10370 and the cetane number as specified in DIN 51773. The determination of the sulfur content can be effected as specified in EN ISO 20884 and that of the chlorine content as specified in DIN 51577-3. Tin, zinc and silicon contents can be measured as specified in DIN 51396-1, and the boron content as specified in DIN 51443-2.

The terms “phosphorus content”, “alkali metal content”, “alkaline earth metal content”, “acid content/acid number”, “iodine number”, “oxidation stability”, “overall contamination”, “kinematic viscosity”, “flashpoint”, “net calorific value”, “carbon residue”, “cetane number”, “sulfur content”, “chlorine content”, and “zinc”, “tin”, “silicon” and “boron” content” which are used within the scope of the present invention are preferably defined as in the relevant standards for determining their magnitude.

The oil obtained from the fruits and/or seeds of oil crops treated in accordance with the invention can be employed in the food sector, for example as edible oil or for the preparation of margarine, in the cosmetics sector, for example as carrier, as lubricant or as energy source, i.e. as combustible or motor fuel. When the oil obtained is used in the food sector, it has optionally to be subjected to further refining steps in order to eliminate any undesired flavors, aroma substances, colors, inedible components and the like.

The oil is preferably employed as combustible or motor fuel.

The oil according to the invention is distinguished, inter alia, by a reduced acid content and/or improved stability to oxidation and/or a reduced phosphorus content and/or a reduced content of alkali metal and especially alkaline earth metal compounds and/or a reduced content of suspended matter and other interfering components in comparison with oils obtained from untreated oil crops. Additionally or alternatively, the oil according to the invention is distinguished by at least one characteristic mentioned under (iv), (v) and (vii) to (xv), for example by a lower iodine number, a lower kinematic viscosity and/or a lower overall contamination and the like (in comparison with oils which have been obtained from plants not treated in accordance with the invention).

The reaction products of the oil preferably take the form of its reaction products with C₁-C₄-alcohols, i.e. the C₁-C₄-alkyl esters of the fatty acids on which the oils are based. Especially preferably, they take the form of the transesterification products of the oil with methanol or ethanol and in particular with methanol, i.e. the form of the methyl or ethyl esters and in particular the methyl esters of the fatty acids on which the oils are based. The C₁-C₄-alkyl esters are obtainable by transesterifying the vegetable oil with a C₁-C₄-alcohol, usually in the presence of a catalyst (generally a base). During this process, the fatty acid triglycerides of the oil are converted into the C₁-C₄-alkyl esters of the fatty acids in question. These esters are referred to as C₁-C₄-alkyl esters of the vegetable oil, for the purposes of the present invention.

The reaction products of the oil and in particular its transesterification products with C₁-C₄-alcohols are especially suitable for use as an energy source, i.e. as motor fuel or combustible.

The reaction products of the oil, and in particular the C₁-C₄-alkyl esters of the oil, are distinguished by the properties mentioned for the oil.

When pressing the fruits and/or seeds of oil crops, the residue obtained is a presscake which, like the fruits and seeds, is distinguished by a reduced content of phosphorus and/or alkali metal and especially alkaline earth metal compounds and/or a reduced acid content and in particular by a reduced phosphorus content and/or acid content. This presscake can be employed not only in the feed sector, but also as a direct source of energy, i.e. as combustible, especially in furnace installations, the use as energy source being preferred.

The oil crop products are especially preferably selected among seeds, vegetable oils and their reaction products, for example the transesterification products with C₁-C₄-alcohols. The oil crop products are, in particular, selected among oils and their reaction products, for example the transesterification products with C₁-C₄-alcohols.

The treatment of oil crops or of the seeds from which they grow with the above-specified fungicides, optionally in combination with growth regulators, makes the plants' development more homogeneous. Thus, for example, flowering within the individual plant stories (i.e. those zones within a plant (one and the same plant) which are on different levels) takes place more simultaneously, i.e. in a significantly narrower interval, as is the case for shoot development and in particular fruit/seed maturation. The same also applies analogously to the development in plants with plant parts which extend along a larger diameter around the stem as the center, for example the seeds in sunflowers. The increase in the quality of the oil crop products which manifests itself for example in a reduction in the phosphorus content and/or the alkali metal content and/or alkaline earth metal content and/or the acid content and/or in the increase in the oxidation stability and the like can probably be attributed to this more homogeneous development of the plant, at least in part. This, and in particular the simultaneous retaining of an advantageous harvest time, gives seeds/fruits of oil crops with an optimal quality with regard to the above criteria. Simultaneously, the quantity is also optimized since the more simultaneous maturation of fruit/seeds at harvest time the fewer fruit/seeds are immature or overripe, which means lower harvest losses occur.

1-33. (canceled)

34. A method of achieving a chronologically more uniform phenological development of an oil crop comprising treating the oil crop or plant parts thereof or seed thereof with at least one fungicide where the at least one fungicide is an arylamide, a heterocyclylamide, a carbamate, a dicarboximide, an azole, a strobilurin, or a morpholine, optionally in combination with at least one growth regulator.

35. The method of claim 34 wherein the phenological development is a more uniform maturation of the seeds of oil crops.

36. The method of claim 34, where the oil crop is selected from the group consisting of oilseed rape, turnip rape, mustard, oil radish, false flax, garden rocket, crambe, sunflower, safflower, thistle, calendula, soybean, lupine, flax, hemp, oil pumpkin, poppy, maize, oil palm and peanut.

37. The method of claim 36, where the oil crop is selected from the group consisting of oilseed rape and turnip rape.

38. The method of claim 34, where the arylamide and the heterocyclylamide are selected from compounds of the formula I
A-CO—NH-M-Q-R¹
in which
A is an aryl group or an aromatic or nonaromatic 5- or 6-membered heterocycle which comprises, as ring members, 1 to 3 heteroatoms or heteroatom-comprising groups selected from the group consisting of O, S, N and NR², R² being hydrogen or C₁-C₈-alkyl, the aryl group or the heterocycle optionally having 1, 2 or 3 substituents which are selected independently of one another from the group consisting of halogen, C₁-C₈-alkyl, C₁-C₈-haloalkyl, C₁-C₈-alkoxy, C₁-C₈-haloalkoxy, C₁-C₈-alkylthio, C₁-C₈-alkylsulfinyl and C₁-C₈alkylsulfonyl;
M is a thienyl ring or a phenyl ring, where the thienyl and the phenyl ring may have attached to them 1, 2 or 3 halogen atoms and where the phenyl ring is optionally fused to a saturated 5-membered ring which is optionally substituted by 1, 2 or 3 C₁-C₈-alkyl groups and/or optionally contains, as ring member, a hetero atom selected from the group consisting of O and S;
Q is a bond, C₁-C₆-alkylene, C₂-C₆-alkenylene, C₂-C₆-alkynylene, C₃-C₆-cycloalkylene, C₃-C₆-cycloalkenylene, —O—C₁-C₆-alkylene, —O—C₂-C₆-alkenylene, —O—C₂-C₆-alkynylene, —O—C₃-C₆-cycloalkylene, —O—C₃-C₆-cycloalkenylene, —S—C₁-C₆-alkylene, —S—C₂-C₆-alkenylene, —S—C₂-C₆-alkynylene, —S—C₃-C₆-cycloalkylene, —S—C₃-C₆-cycloalkenylene, —SO—C₁-C₆-alkylene, —SO—C₂-C₆-alkenylene, —SO—C₂-C₆-alkynylene, —SO—C₃-C₆-cycloalkylene, —SO—C₃-C₆-cycloalkenylene, —SO₂—C₁-C₆-alkylene, —SO₂—C₂-C₆-alkenylene, —SO₂—C₂-C₆-alkynylene, —SO₂—C₃-C₆-cycloalkylene, —SO₂—C₃-C₆-cycloalkenylene, O, S, SO or SO₂;
where the aliphatic and cycloaliphatic radicals in Q may be partially or fully halogenated and/or the cycloaliphatic radicals may be substituted by 1, 2 or 3 C₁-C₈-alkyl radicals;
R′ is hydrogen, halogen, C₃-C₆-cycloalkyl or phenyl, where the cycloalkyl radical may have attached to it a methyl group and where phenyl may be substituted by 1 to 5 halogen atoms and/or by 1, 2 or 3 substituents which are selected independently of one another from the group consisting of C₁-C₈-alkyl, C₁-C₈-haloalkyl, C₁-C₈-alkoxy, C₁-C₈-haloalkoxy, C₁-C₈-alkylthio and C₁-C₈-haloalkylthio.

39. The method of claim 38, where the amide of the formula I is selected from anilides of the formula I.1 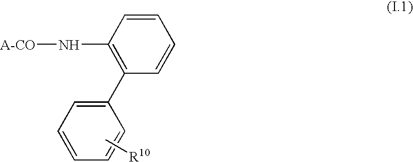
in which A is a group of the formula A1 to A8 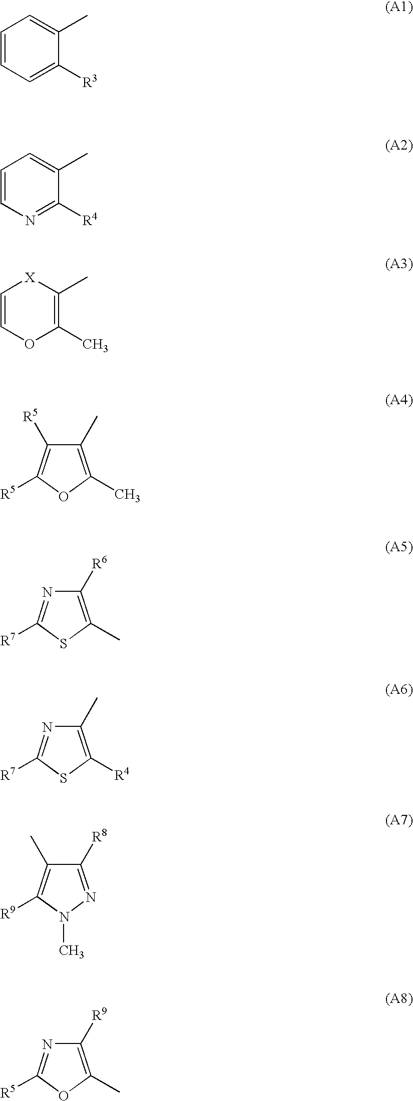
in which
X is CH₂, S, SO or SO₂;
R³ is CH₃, CHF₂, CF₃, Cl, Br or I;
R⁴ is CF₃ or Cl;
R⁵ is hydrogen or CH₃;
R⁶ is CH₃, CHF₂, CF₃ or Cl;
R⁷ is hydrogen, CH₃ or Cl;
R⁸ is CH₃, CHF₂ or CF₃;
R⁹ is hydrogen, CH₃, CHF₂, CF₃ or Cl; and
R¹⁰ is C₁-C₄-alkyl, C₁-C₄-alkoxy, C₁-C₄-alkylthio or halogen.

40. The method of claim 39, where A is the group A2, in which R⁴ is halogen and R¹⁰ is halogen.

41. The method of claim 40, wherein the amide I is selected from anilides of the formulae I.1.1 and I.1.2

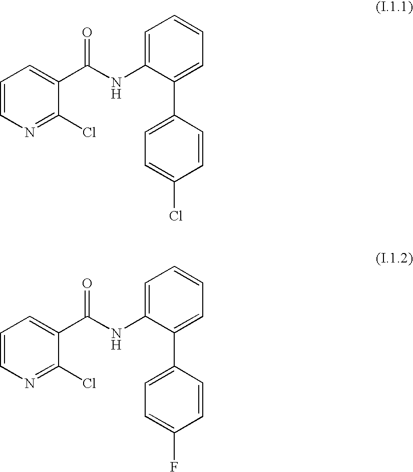

42. The method of claim 34, wherein the azole is selected from the group consisting of difenoconazole, flusilazol, metconazole, paclobutrazol, prothioconazole and tebuconazole.

43. The method of claim 34, wherein the strobilurin is selected from the group consisting of azoxystrobin, dimoxystrobin and pyraclostrobin.

44. The method of claim 34, wherein the morpholine fungicide is dimethomorph.

45. The method of claim 34, wherein at least one arylamide or heterocyclylamide is combined with at least one azole.

46. The method of claim 45, where the arylamide or heterocyclylamide employed is boscalid and the azole employed is metconazole.

47. The method of claim 34, wherein the growth regulators are selected from
(a) acylcyclohexanediones of the formula (IV) 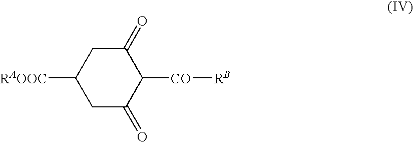
in which
R^{A} is H or C₁-C₁₀-alkyl and
R^{B} is C₁-C₁₀-alkyl or C₃-C₁₀-cycloalkyl
or salts thereof; and
(b) quaternary ammonium compounds of the formula (V) 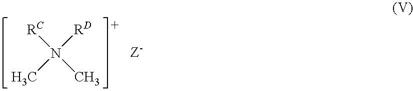
in which
R^{C} and R^{D} independently of one another are C₁-C₁₀-alkyl which is optionally substituted by at least one halogen atom, or a C₃-C₁₀-cycloalkyl; or
R^{C} and R^{D} together form a bridging unit —(CH₂)ₙ—, —(CH₂)₂—O—(CH₂)₂— or —(CH₂)—CH═CH—(CH₂)—NH—,
in which n is 4 or 5, and
Z⁻ is a counter anion which is selected from the group consisting of halide ions, sulfate ions, C₁-C₁₀-alkylsulfonate ions, borate ions, and carbonate ions and mixtures of thereof.

48. The method of claim 47, wherein the compounds of the formula (IV) are the alkali metal or alkaline earth metal salts thereof in which R^{A} is H.

49. The method of claim 48, wherein R^{B} is ethyl.

50. The method of claim 48, which is the calcium salt.

51. The method of claim 37, wherein, in compounds of the formula (IV), R^{A} is ethyl and R^{B} is cyclopropyl.

52. The method of claim 47, wherein, in compounds of the formula (V), R^{C} is methyl and R^{D} is 2-chloroethyl.

53. The method of claim 47, wherein, in compounds of the formula (V), R^{C} and R^{D} together form a bridging unit —(CH₂)₅—.

54. The method of claim 47, wherein, in compounds of the formula (V) Z⁻ is chloride.

55. A method of achieving a chronologically more uniform course of the phenological development of oil crops, where the oil crop or plant parts thereof or seed thereof is treated with at least one fungicide, optionally in combination with at least one growth regulator selected from
(a) acylcyclohexanediones of the formula (IV) 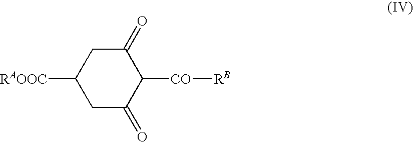
in which
R^{A} is H or C₁-C₁₀-alkyl and
R^{B} is C₁-C₁₀-alkyl or C₃-C₁₀-cycloalkyl or salts thereof; and
(b) quaternary ammonium compounds of the formula (V) 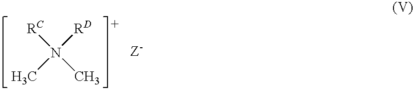
in which
R^{C} and R^{D} independently of one another are C₁-C₁₀-alkyl which is optionally substituted by at least one halogen atom, or a C₃-C₁₀-cycloalkyl; or
R^{C} and R^{D} together form a bridging unit —(CH₂)ₙ—, —(CH₂)₂—O—(CH₂)₂— or —(CH₂)—CH═CH—(CH₂)—NH—,
in which n is 4 or 5, and
Z⁻ is a counter anion which is selected from the group consisting of halide ions, sulfate ions, C₁-C₁₀-alkylsulfonate ions, borate ions, carbonate ions and mixtures of these.

56. The method according to claim 55, where the oil crop or plant parts thereof are treated with at least one aryl- or heterocyclylamide in combination with at least one azole.

57. The method according to claim 56, where the oil crop or plant parts thereof are treated with the at least one azole before anthesis and with the at least one aryl- or heterocyclylamide during anthesis.

58. A method of increasing the quality and optionally the quantity of oil crop products comprising treating an oil crop or plant parts thereof or its seed with at least one fungicide, optionally in combination with at least one growth regulator, harvesting the seed of the oil crop when their water content amounts to no more than 15% by weight based on the total weight of the seed, and obtaining the oil crop product, the increase in quality being selected from the following criteria:
(i) reducing the phosphorus content of at least one oil crop product;
(ii) reducing the alkali and/or alkaline earth metal content of at least one oil crop product;
(iii) increasing the oxidation stability of at least one oil crop product;
(iv) reducing the overall contamination of at least one oil crop product;
(v) lowering the iodine number of at least one oil crop product;
(vi) lowering the acid number of at least one oil crop product;
(vii) reducing the kinematic viscosity of at least one oil crop product;
(viii) reducing the sulfuric content of at least one oil crop product;
(ix) increasing the flashpoint of at least one oil crop product;
(x) increasing the net calorific value of at least one oil crop product;
(xi) reducing the carbon residue of at least one oil crop product;
(xii) increasing the cetane number of at least one oil crop product;
(xiii) reducing the nitrogen content of at least one oil crop product;
(xiv) reducing the chlorine content of at least one oil crop product; and
(xv) reducing the tin, zinc, silicon and/or boron content of at least one oil crop product.

59. The method of claim 58, wherein the growth regulator is selected from
(a) acylcyclohexanediones of the formula (IV) 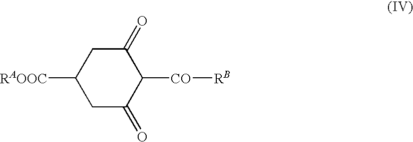
in which
R^{A} is H or C₁-C₁₀-alkyl and
R^{B} is C₁-C₁₀-alkyl or C₃-C₁₀-cycloalkyl
or salts thereof; and
(b) quaternary ammonium compounds of the formula (V) 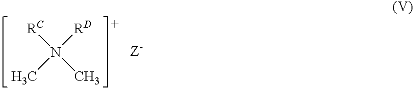
in which
R^{C} and R^{D} independently of one another are C₁-C₁₀-alkyl which is optionally substituted by at least one halogen atom, or a C₃-C₁₀-cycloalkyl; or
R^{C} and R^{D} together form a bridging unit —(CH₂)ₙ—, —(CH₂)₂—O—(CH₂)₂— or —(CH₂)—CH═CH—(CH₂)—NH—,
in which n is 4 or 5, and
Z⁻ is a counter anion which is selected from halide ions, sulfate ions, C₁-C₁₀-alkylsulfonate ions, borate ions, carbonate ions and mixtures of these.

60. The method according to claim 59, wherein the oil crop products are selected from the group consisting of the fruits, seeds, presscakes, oil and reaction products of the oil which have been obtained from the oil crops.

61. The method according to claim 60, wherein the reaction products of the oil are the transesterification products of the oil with C₁-C₄-alcohols.

62. The method according to claim 59, wherein the oil crop products are selected from the oil obtained from the oil crops and its reaction products.

63. The method according to claim 59, the oil crops being selected from the group consisting of oilseed rape, turnip rape, mustard, oil radish, false flax, garden rocket, crambe, sunflower, safflower, thistle, calendula, soybean, lupine, flax, hemp, oil pumpkin, poppy, maize, oil palm and peanut.

64. The method according to claim 63, wherein the oil crops are selected from the group consisting of oilseed rape, turnip rape, sunflower and maize.

65. The method according to claim 64, wherein the oil crops are selected from the group consisting of oilseed rape and turnip rape.

66. The method according to claim 59, wherein the seeds of the oil crop plant are harvested when their water content is 5 to 15% by weight based on the total seed weight.

67. The method according to claim 65, wherein the seeds of the oilseed rape or turnip rape plant are harvested when their water content is no more than 10% by weight based on the total seed weight.
